(19) United States
(12) Patent Application Publication
(10) Pub. No.: US 2011/0082147 A1
(43) Pub. Date: Apr. 7, 2011

(54) SUBSTITUTED IMIDAZOTRIAZINES

(75) Inventors: Scott L. Harbeson, Cambridge, MA (US); Roger D. Tung, Lexington, MA (US); Julie F. Liu, Lexington, MA (US)

(73) Assignee: CoNCERT Pharmaceuticals, Inc., Lexington, MA (US)

(21) Appl. No.: 12/842,589

(22) Filed: Jul. 23, 2010

Related U.S. Application Data
(60) Provisional application No. 61/237,939, filed on Aug. 28, 2009.
(60) Provisional application No. 61/271,695, filed on Jul. 24, 2009.

(51) Int. Cl. A61K 31/53; C07D 487/04; A61P 25/22; A61P 25/08; A61P 25/00; A61P 29/00; A61P 25/06

(52) U.S. Cl. 514/243; 544/184

(57) ABSTRACT

This invention relates to novel substituted imidazotriazines and pharmaceutically acceptable salts thereof. This invention also provides compositions comprising a compound of this invention and the use of such compositions in methods of treating diseases and conditions that are beneficially treated by administering a compound showing partial-agonist activity at the GABA α2, α3 and α5 subtype receptors, and antagonist activity at the al subtype receptor.

### RELATED APPLICATION

This application claims the benefit of U.S. Provisional Application No. 61/237,939 filed on Aug. 28, 2009, and U.S. Provisional Application No. 61/271,695, filed on Jul. 24, 2009.

The entire teachings of the above applications are incorporated herein by reference.

### BACKGROUND OF THE INVENTION

Many current medicines suffer from poor absorption, distribution, metabolism and/or excretion (ADME) properties that prevent their wider use or limit their use in certain indications. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment. A rapidly metabolized drug may also expose patients to undesirable toxic or reactive metabolites.

Another ADME limitation that affects many medicines is the formation of toxic or biologically reactive metabolites. As a result, some patients receiving the drug may experience toxicities, or the safe dosing of such drugs may be limited such that patients receive a suboptimal amount of the active agent. In certain cases, modifying dosing intervals or formulation approaches can help to reduce clinical adverse effects, but often the formation of such undesirable metabolites is intrinsic to the metabolism of the compound.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J. et al., Antimicrobial agents and chemotherapy, 1997, 41(3): 654-60). Ritonavir, however, causes adverse effects and adds to the pill burden for HIV patients who must already take a combination of different drugs. Similarly, the CYP2D6 inhibitor quinidine has been added to dextromethorphan for the purpose of reducing rapid CYP2D6 metabolism of dextromethorphan in a treatment of pseudobulbar affect. Quinidine, however, has unwanted side effects that greatly limit its use in potential combination therapy (see Wang, L et al., Clinical Pharmacology and Therapeutics, 1994, 56(6 Pt 1): 659-67; and FDA label for quinidine at www.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug or to reduce the formation of undesirable metabolites by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

GABA_{A} receptors are ligand-gated chloride channels that mediate the inhibitory effects of γ-aminobutyric acid (GABA) in the central nervous system. GABA_{A} receptors are heteromeric proteins of five subunits primarily found as receptors containing α, β, and γ subunits in a 2:2:1 stoichiometry. GABA_{A} receptors containing the α1, α2, α3, or α5 subunits contain a binding site for benzodiazepines, which is the basis for the pharmacologic activity of benzodiazepines.

TPA-023B, also known as 6,2′-difluoro-5′-[3-(1-hydroxy-1-methylethyl)imidazo[1,2-b][1,2,4]triazin-7-yl]biphenyl-2-carbonitrile and as 2′,6-difluoro-5′-(3-(2-hydroxypropan-2-yl)imidazo[1,2-b][1,2,4]triazin-7-yl)biphenyl-2-carbonitrile, is a selective ligand for GABA_{A} receptors, showing in vitro partial-agonist activity at the α2, α3 and α5 subtype receptors, and antagonist activity at the al subtype receptor. Such agents are of benefit in the treatment and/or prevention of disorders of the central nervous system, including anxiety and convulsions in addition to prevention and treatment of neuropathic, inflammatory and migraine pain.

The pharmacokinetic properties of TPA-023B have been evaluated in humans in a phase I clinical trial. Compounds exhibiting a GABA_{A} selectivity similar to TPA-023B have also shown activity in pharmacological models of inflammatory and neuropathic pain.

Despite the purported beneficial activities of TPA-023B, there is a continuing need for new compounds that have beneficial effects as anxiolytics and antinociceptives without sedative and proconvulsant effects.

### SUMMARY OF THE INVENTION

This invention relates to novel substituted imidazotriazines and pharmaceutically acceptable salts thereof. This invention also provides compositions comprising a compound of this invention and the use of such compositions in methods of treating diseases and conditions that are beneficially treated by administering an antagonist for GABA_{A} receptors at the benzodiazepine site consisting of an al subunit, or a partial agonist for GABA_{A} receptors at the benzodiazepine site containing an α2 or α3 or α5 subunit.

### DETAILED DESCRIPTION OF THE INVENTION

The term “treat” means decrease, suppress, attenuate, diminish, arrest, or stabilize the development or progression of a disease (e.g., a disease or disorder delineated herein).

“Disease” means any condition or disorder that damages or interferes with the normal function of a cell, tissue, or organ.

The term “alkyl” refers to a monovalent saturated hydrocarbon group. C₁-C₆ alkyl is an alkyl having from 1 to 6 carbon atoms. An alkyl may be linear or branched. Examples of alkyl groups include methyl; ethyl; propyl, including n-propyl and isopropyl; butyl, including n-butyl, isobutyl, sec-butyl, and t-butyl; pentyl, including, for example, n-pentyl, isopentyl, and neopentyl; and hexyl, including, for example, n-hexyl and 2-methylpentyl.

The term “aryl” refers to a monovalent aromatic carbocyclic ring system, which may be a monocyclic, fused bicyclic, or fused tricyclic ring system. The term “C₆-C₁₄ aryl” refers to an aryl having from 6 to 14 ring carbon atoms. An example of C₆-C₁₄ aryl is C₆-C₁₀ aryl. More particular examples of aryl groups include phenyl, naphthyl, anthracyl, and phenanthryl.

The term “heteroaryl” refers to a monovalent aromatic ring system wherein from 1 to 4 ring atoms are heteroatoms independently selected from the group consisting of O, N and S, and having from 5 to 14 ring atoms. The ring system may be a monocyclic, fused bicyclic, or fused tricyclic ring system. The term “5 to 14-membered heteroaryl” refers to a heteroaryl wherein the number of ring atoms is from 5 to 14. Examples of 5 to 14-membered heteroaryl include 5 to 10-membered heteroaryl and 5 to 6-membered heteroaryl. More particular examples of heteroaryl groups include furanyl, furazanyl, imidazolinyl, isothiazolyl, isoxazolyl, oxadiazolyl, oxazolyl, pyrimidinyl, phenanthridinyl, pyrazinyl, pyrazolinyl, pyrazolyl, pyridazinyl, pyridooxazolyl, pyridoimidazolyl, pyridothiazolyl, pyridinyl, pyrimidinyl, pyrrolinyl, thiadiazinyl, thiadiazolyl, thienyl, thienothiazolyl, thienooxazolyl, thienoimidazolyl, thiophenyl, triazinyl, and triazolyl.

The term “halogen” refers to fluorine, chlorine, bromine or iodine.

It will be recognized that some variation of natural isotopic abundance occurs in a synthesized compound depending upon the origin of chemical materials used in the synthesis. Thus, a preparation of TPA-023B will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66:15; Gannes L Z et al., Comp Biochem Physiol Mol Integr Physiol 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species in which the chemical structure differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free of another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes specified herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers. “Tert” and “t-” each refer to tertiary. “US” refers to the United States of America.

The phrase “substituted with deuterium” means that one or more positions in the indicated moiety are substituted with a deuterium atom.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula I:

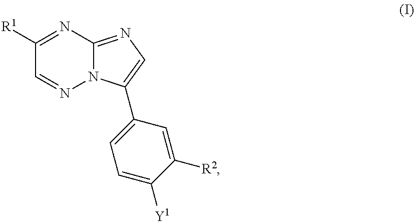

or a pharmaceutically acceptable salt thereof, wherein:

R¹ is
(a) C₁-C₆ alkyl that is optionally substituted with one or two groups selected from halogen and —OH;
(b) —OC₁-C₆ alkyl;
(c) —C(O)H;
(d) —C(O)C₁-C₆ alkyl;
(e) —C(O)OC₁-C₆ alkyl; or
(f) —CR³═NOR⁴,
wherein R¹ is optionally substituted with one or more deuterium;

R² is aryl or heteroaryl, wherein R² is optionally substituted with one or two groups independently selected from the group consisting of halogen, —OCH₃, —OCD₃, —CH₂OH, —CD₂OH, —CH₃, —CD₃, —CH₂CH₃, —CD₂CH₃, —CH₂CD₃, —CD₂CD₃, —CF₃, —CN, —C(O)H, —C(O)OCH₃, —NH₂, —C(O)CH₃, —C(O)CD₃, —SCH₃, —SCD₃, —S(O)CH₃, —S(O)CD₃, —S(O₂)CH₃, —S(O₂)CD₃, and —CH═NOH;

R³ is selected from hydrogen, deuterium, and C₁-C₆ alkyl that is optionally substituted with one or more deuterium;

R⁴ is C₁-C₆ alkyl that is optionally substituted with one or more hydroxyl or with one or more —N(C₁-C₆ alkyl)₂ wherein each alkyl in R⁴ is optionally substituted with one or more deuterium; and

Y¹ is hydrogen, —Cl, or —F;

with the proviso that at least one of R¹ and R² comprises deuterium.

In one embodiment, R¹ is selected from methyl, fluoromethyl, difluoromethyl, hydroxymethyl, hydroxyethyl, difluoroethyl, fluoropropyl, hydroxypropyl, t-butyl, O-methyl, —C(O)H, —C(O)methyl, -carbonyloxymethyl and —CR³═NOR⁴, wherein R¹ is optionally substituted with one or more deuterium. Specific examples of R¹ include —CH₃, —CD₃, —CF₂CH₃, —CF₂CD₃, —CF(CH₃)₂, —CF(CD₃)₂, —C(OH)(CH₃)₂, —C(OH)(CD₃)₂, —C(CH₃)₃, and —C(CD₃)₃.

In one embodiment, R² is selected from the group consisting of phenyl, pyridazinyl, pyrimidinyl, pyrazinyl, furyl, pyrrolyl, pyrazolyl, oxazolyl, isoxazolyl, imidazolyl, oxadiazolyl, thiadiazolyl, triazolyl, tetrazolyl, pyridyl, thienyl, and thiazolyl wherein R² is optionally substituted with one or two groups independently selected from the group consisting of —F, —OCH₃, —OCD₃, —CH₂OH, —CD₂OH, —CH₃, —CD₃, —CH₂CH₃, —CD₂CH₃, —CH₂CD₃, —CD₂CD₃, —Cl, —CF₃, —CN, —C(O)H, —C(O)OCH₃, —NH₂, —C(O)CH₃, —C(O)CD₃, —SCH₃, —SCD₃, —S(O)CH₃, —S(O)CD₃, and —CH═NOH. In an example of this embodiment, R² is phenyl, pyridyl, thienyl, or thiazolyl wherein R² is optionally substituted with one or two groups selected from —F, —OCH₃, —OCD₃, —CH₃, —CD₃, —CH₂CH₃, —CD₂CH₃, —CH₂CD₃, —CD₂CD₃, —CF₃, —CN, and —C(O)H.

In one embodiment, R³ is selected from hydrogen, deuterium, —CH₃, and —CD₃.

In one embodiment, R⁴ is selected from methyl, ethyl, hydroxyethyl, and (dimethylamino)ethyl wherein each alkyl in R⁴ is optionally substituted with one or more deuterium.

In one embodiment, R² is phenyl, pyridyl, thienyl, or thiazolyl wherein R² is optionally substituted with one or two groups selected from —F, —OCH₃, —OCD₃, —CH₃, —CD₃, —CH₂CH₃, —CD₂CH₃, —CH₂CD₃, —CD₂CD₃, —CF₃, —CN and —C(O)H; R³ is selected from hydrogen, deuterium, —CH₃, and —CD₃; and R⁴ is selected from methyl, ethyl, hydroxyethyl, and (dimethylamino)ethyl wherein each alkyl in R⁴ is optionally substituted with one or more deuterium.

In one embodiment, Y¹ is hydrogen or —F. In an example of this embodiment, R¹ is selected from —CH₃, —CD₃, —CF₂CH₃, —CF₂CD₃, —CF(CH₃)₂, —CF(CD₃)₂, —C(OH)(CH₃)₂, —C(OH)(CD₃)₂, —C(CH₃)₃, and —C(CD₃)₃, and R² is phenyl, pyridyl, thienyl, or thiazolyl wherein R² is optionally substituted with one or two groups selected from —F, —OCH₃, —OCD₃, —CH₃, —CD₃, —CH₂CH₃, —CD₂CH₃, —CH₂CD₃, —CD₂CD₃, —CF₃, —CN, and —C(O)H.

In one embodiment of this invention the compound of Formula I is a compound of Formula Ia:

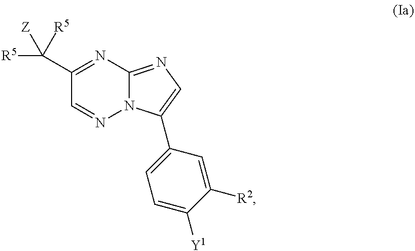

or a pharmaceutically acceptable salt thereof, wherein:

R² is defined as in Formula I;

Z is —OH or methyl, wherein the methyl of Z is optionally substituted with one or more deuterium;

each R⁵ is methyl wherein each R⁵ is optionally independently substituted with one or more deuterium; and

Y¹ is hydrogen or —F;

with the proviso that if each R⁵ is not substituted with deuterium; and Z is not substituted with deuterium; then R² comprises deuterium.

One embodiment of this invention provides a compound of Formula Ia, wherein —CZ(R⁵)₂ is —C(CH₃)₃ or —C(CD₃)₃. In one aspect of this embodiment, Y¹ is hydrogen. In another aspect, Y¹ is —F.

One embodiment provides compounds of Formula Ia, wherein —CZ(R⁵)₂ is —C(OH)(CD₃)₂. In one aspect of this embodiment, Y¹ is hydrogen. In another aspect, Y¹ is —F.

One embodiment provides compounds of Formula Ia, wherein —CZ(R⁵)₂ is —C(OH)(CH₃)₂. In one aspect of this embodiment, Y¹ is hydrogen. In another aspect, Y¹ is —F.

One embodiment of the invention provides a compound of Formula Ia wherein —CZ(R⁵)₂ is —C(CH₃)₃, —C(CD₃)₃, —C(CH₃)₂OH, or —C(CD₃)₂OH. In one aspect of this embodiment, —CZ(R⁵)₂ is —C(CD₃)₃ or —C(OH)(CD₃)₂. As an example, —CZ(R⁵)₂ may be —C(CD₃)₃. As another example, —CZ(R⁵)₂ may be —C(OH)(CD₃)₂. In one aspect of this embodiment, R² is phenyl or pyridyl, wherein R² is optionally substituted as defined in Formula Ia. In one aspect of this embodiment, Y¹ is hydrogen. In another aspect, Y¹ is —F.

One embodiment provides a compound of Formula Ia wherein R² is phenyl or pyridyl, wherein R² is optionally substituted as defined in Formula Ia. In one aspect of this embodiment, Y¹ is hydrogen. In another aspect, Y¹ is —F. In one aspect of this embodiment, R² is phenyl optionally substituted with one or two groups independently selected from —CH₃, —CD₃, —CN, and —F. As an example of this aspect, R² may be phenyl optionally substituted with one or two groups independently selected from —CN, and —F. As an example, R² may be

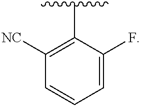

In another aspect of this embodiment, R² is pyridyl optionally substituted with one or two groups independently selected from —CH₃, —CD₃, —CN, and —F. In one example of this aspect, the pyridyl nitrogen is ortho or meta relative to the point of attachment of R² to the imidazotriazinyl of Formula Ia. For example, R² may be selected from:

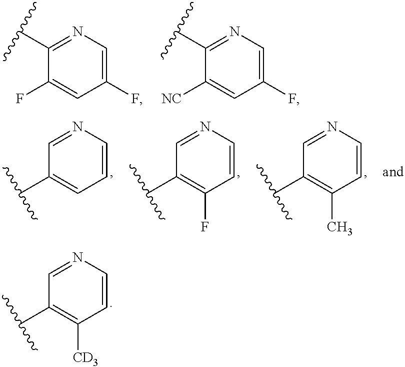

In one embodiment of the compound of Formula Ia, Y¹ is hydrogen. In another embodiment of the compound of Formula Ia, Y¹ is —F.

One embodiment provides a compound of Formula Ia selected from any one of the following compounds:

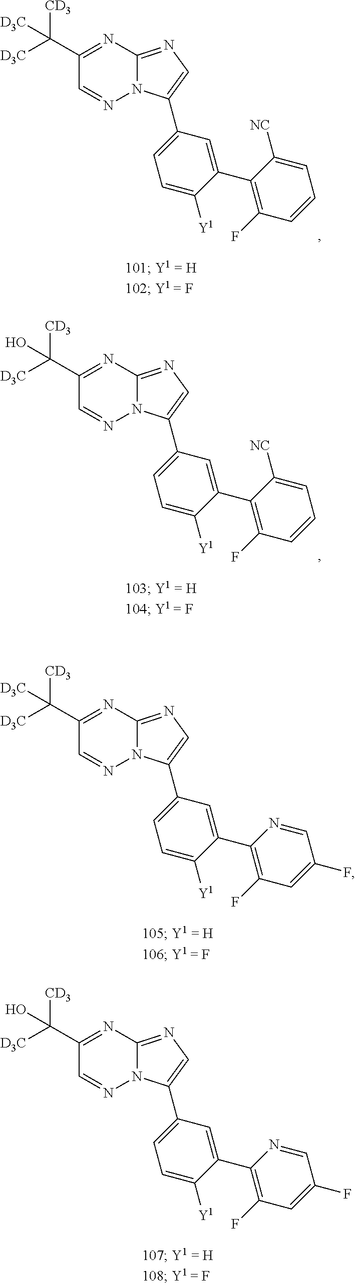

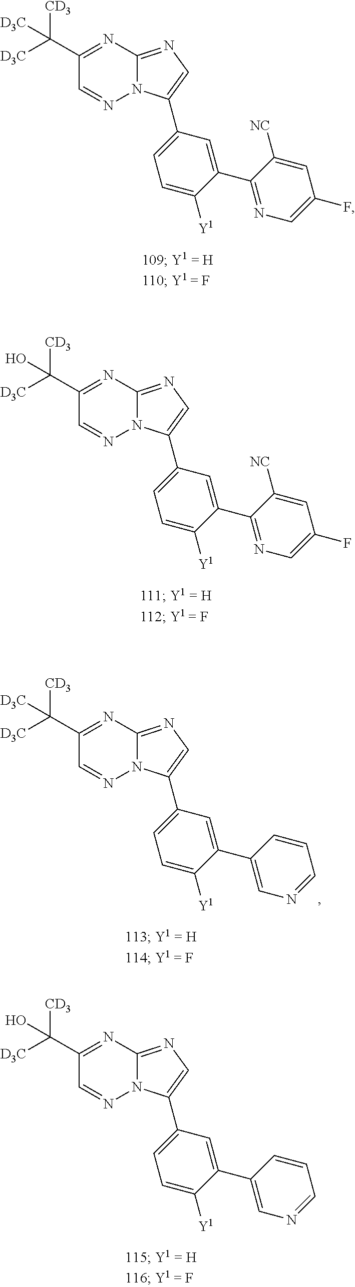

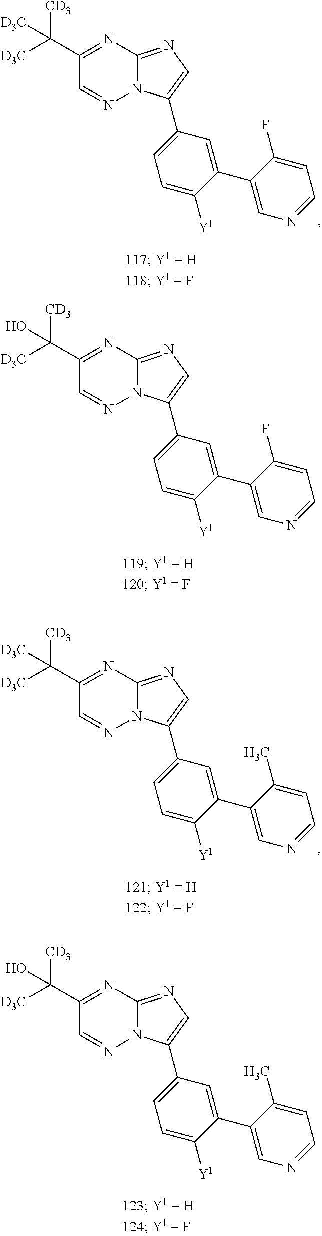

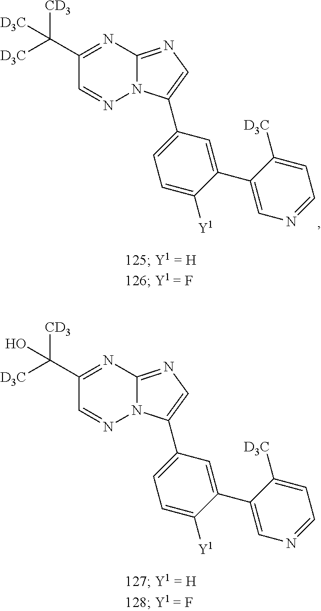

or a pharmaceutically acceptable salt of any of the foregoing.

A number of novel intermediates are described herein that can be used to prepare compounds of Formula I. Such compounds that are useful intermediates include the following:

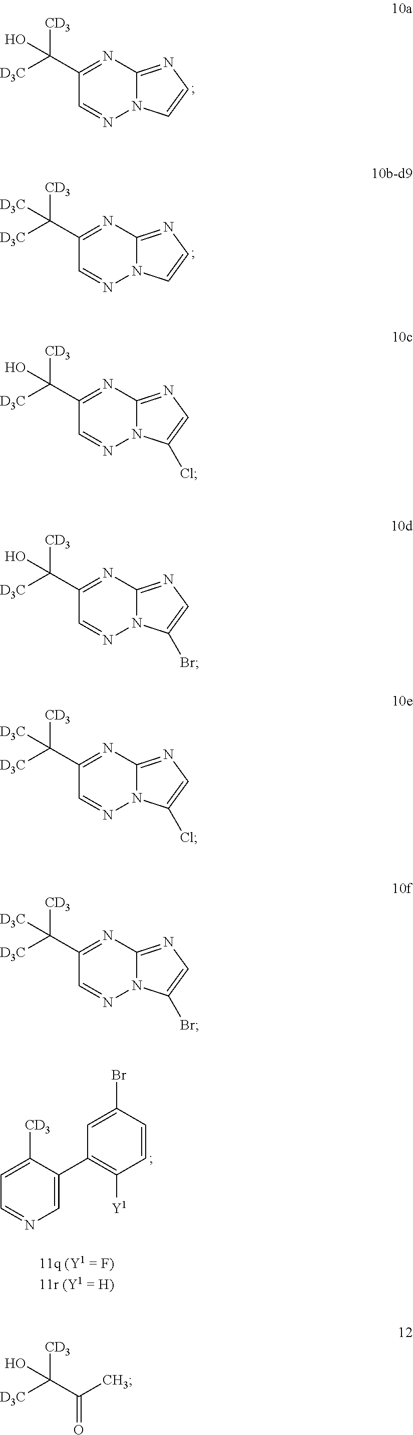

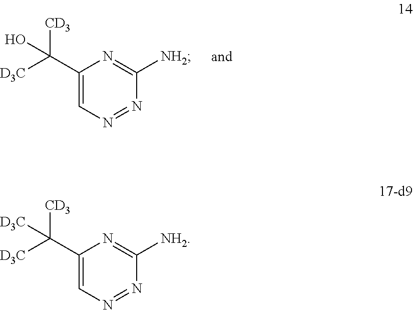

The present invention also provides the compound of formula II:

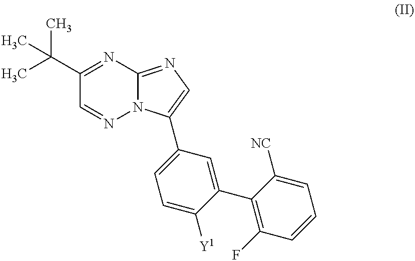

or a pharmaceutically acceptable salt thereof,

wherein Y¹ is hydrogen or —F.

In one embodiment, of formula II, Y¹ is hydrogen. In another embodiment, Y¹ is —F. In another embodiment, the compound of Formula II is Compound 130

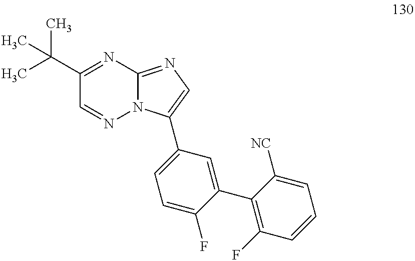

or a pharmaceutically acceptable salt thereof

In another set of embodiments, any atom not designated as deuterium in any of the embodiments set forth above is present at its natural isotopic abundance.

The synthesis of compounds of the invention can be readily achieved by synthetic chemists of ordinary skill by reference to the Exemplary Synthesis and Examples disclosed herein.

### Exemplary Synthesis

Compounds of Formula I may be prepared according to the schemes shown below. Procedures for the synthesis of the natural abundance compounds of Formula I have been disclosed in Gauthier, D R et al., J. Org. Chem. 2005, 70: 5938-5945; WO2002/038568; and WO2003/008418.

A general method for synthesizing compounds of Formula I is depicted in Scheme 1 below.

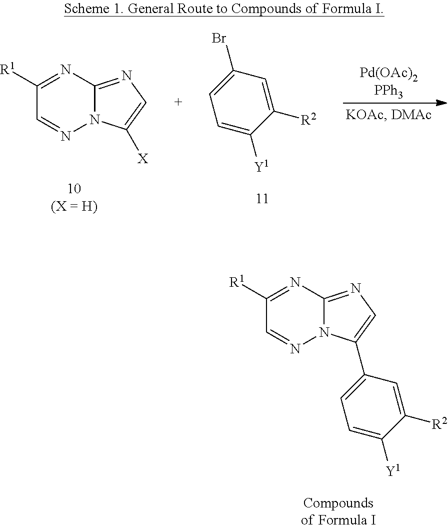

As depicted in Scheme 1, the appropriate imidazotriazine 10 may be coupled to biarylbromide 11 via palladium-catalyzed arylation to provide compounds of Formula I (for example, compounds 101 to 128). Biarylbromides 11a (for the preparation of compounds 106 and 108) and 11b (for the preparation of compounds 113 and 115) are commercially available (see below). For the synthesis of other examples of biarylbromides 11 see Schemes 5a-5j below.

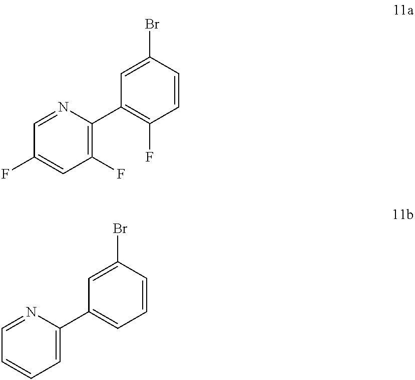

Intermediates 10a and 10b, for use in Scheme 1, may be prepared as depicted in Schemes 2a and 2b below. An alternative method to that shown in Scheme 1 for the synthesis of compounds of Formula I and intermediates 10c, 10d, 10e and 10f is generally described in WO 2003008418. These intermediates may be prepared as generally described through chlorination or bromination of 10b or the appropriately protected 10a.

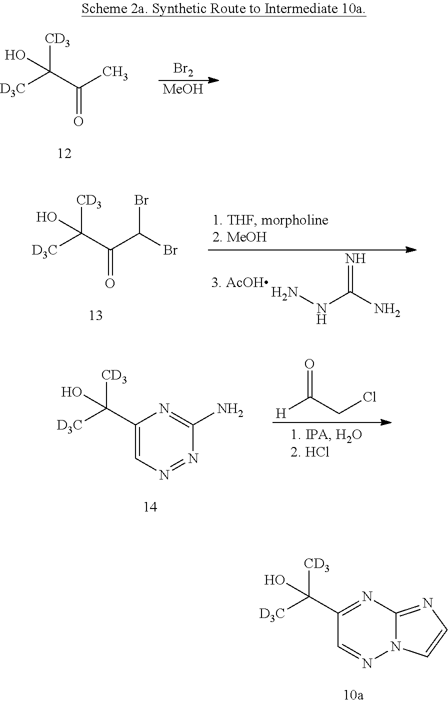

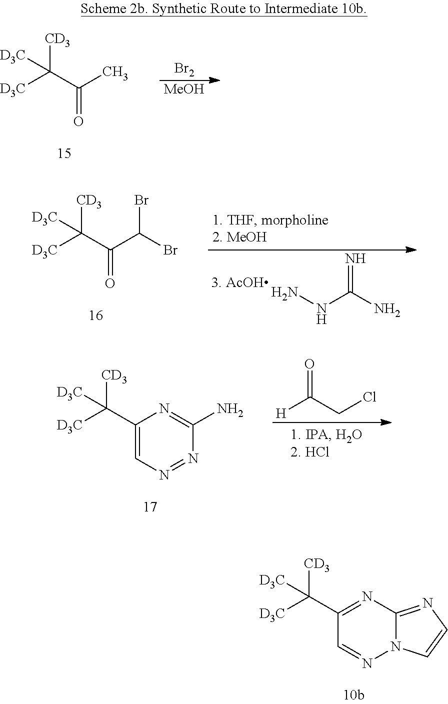

The imidazotriazine intermediates 10a and 10b may be prepared as shown in Schemes 2a and 2b according to the general methods disclosed in Gauthier, D R et al., J. Org. Chem. 2005, 70: 5938-5945.

Intermediate 12, for use in Scheme 2a, may be prepared as depicted in Scheme 3 below.

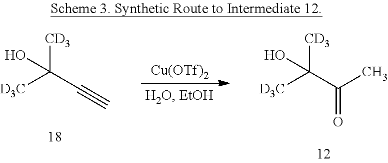

Deuterated ketone 12 may be prepared according to the method shown in Scheme 3 as generally described by Meier, I K et al., J. Mol. Catal., 1993, 78(1): 31-42. Deuterated acetylene 18 is commercially available.

Intermediate 15, for use in Scheme 2b, may be prepared as depicted in Scheme 4 below.

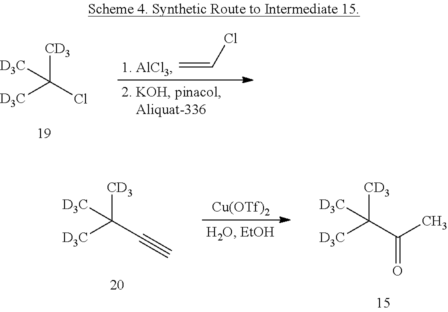

Deuterated ketone 15 may be prepared from commercially available deuterated t-butyl chloride 19 as generally described by Verkruijsse, et al. Synth. Comm. 1990, 20(21), 3355-8. Lewis acid catalyzed addition of 19 to vinyl chloride followed by treatment with KOH affords acetylene 20 which can then be converted to ketone 15 in the presence of copper (II) triflate (Cu(OTf)₂) as generally described by Meier, et al. J. Mol. Catal. 1993, 31-42.

Intermediate 11c may be prepared as depicted in Scheme 5a below.

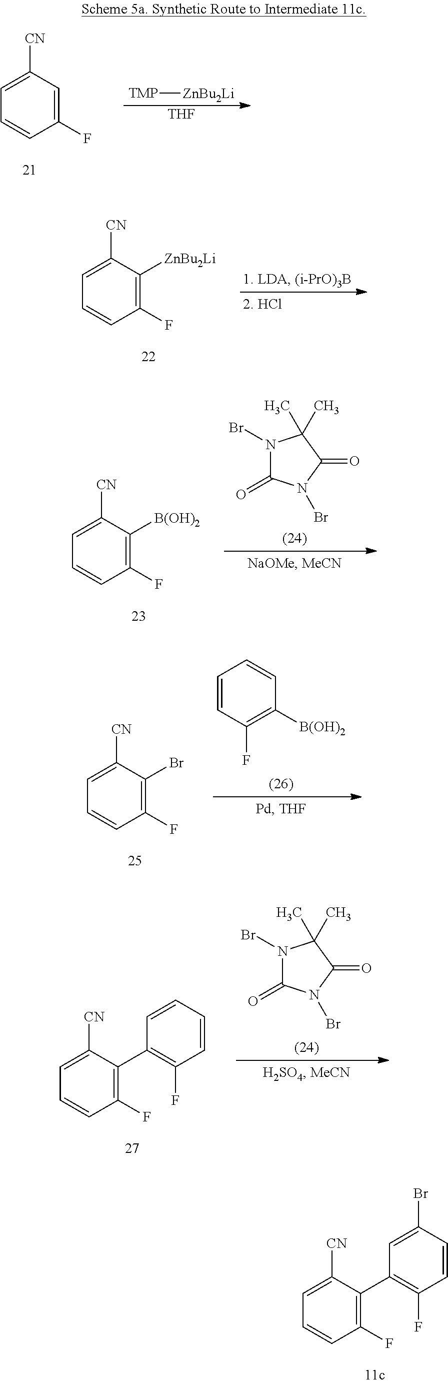

Biarylbromide 11c, for use in the preparation of compounds 102 and 104, may be prepared from the commercially available benzonitrile 21 according to the methods shown in Scheme 5 and disclosed by Gauthier, D R; et al., J. Org. Chem. 2005, 70: 5938-5945. Thus, benzonitrile 21 is treated with lithium dialkyl-2,2,6,6-tetramethylpiperidinozincate (TMP—ZnBu₂Li) to generate the aryl-zincate intermediate 22. Intermediate 22 is then converted to boronic acid 23 which is brominated via treatment with dibromodimethylhydantoin (24) to afford bromide 25. Palladium-catalyzed coupling with commercially-available 2-fluorophenylboronic acid (26) provides biaryl intermediate 27. Bromination with dibromodimethylhydantoin (24) yields biarylbromide 11c.

Intermediate 11d may be prepared as depicted in Scheme 5b below.

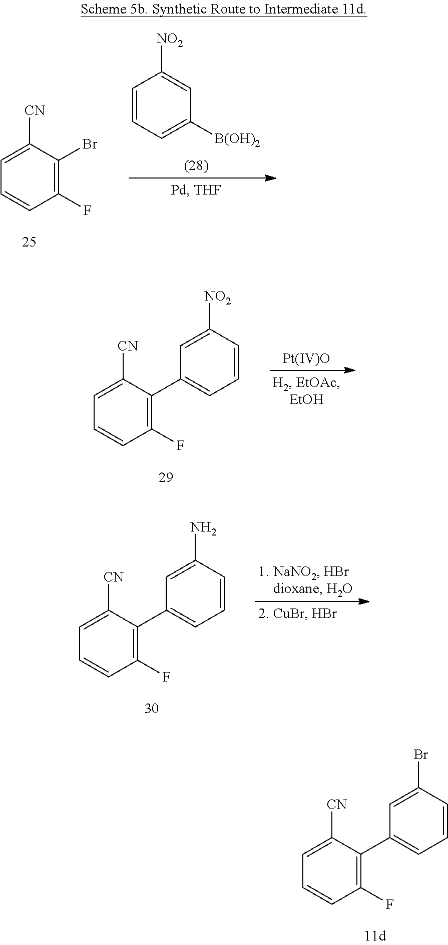

Biarylbromide 11d, for use in the preparation of compounds 101 and 103, may be prepared from the commercially available nitrile 25 and 3-nitrophenylboronic acid (28) according to the method shown in Scheme 5b and disclosed in WO2003008418.

Intermediate 11e may be prepared as depicted in Scheme 5c below.

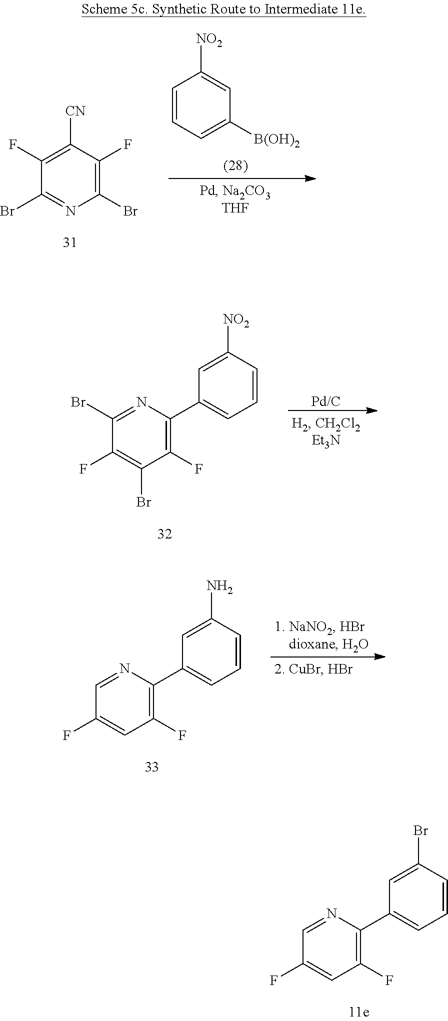

Biarylbromide 11e, for use in the preparation of compounds 105 and 107, may be prepared from the commercially available tribromopyridine 31 and 3-nitrophenylboronic acid (28) according to the methods shown in Scheme 5c and disclosed in WO2002038568.

Intermediates 11f and 11i may be prepared as depicted in Scheme 5d below.

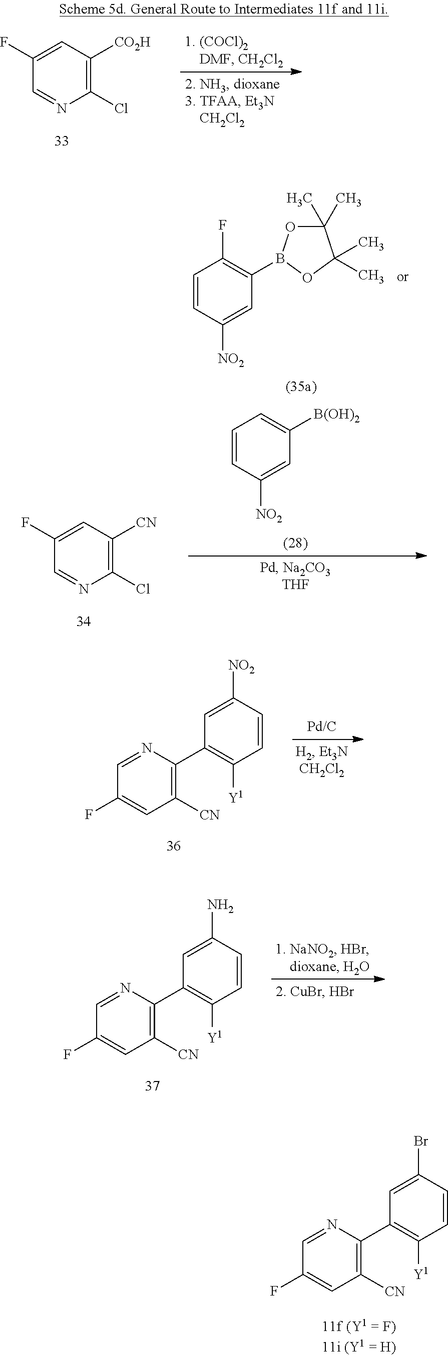

Biarylbromide 11f (Y¹═F), for use in the preparation of compounds 110 and 112, may be prepared from commercially available 2-chloro-5-fluoronicotinic acid 33 according to the method shown in Scheme 5d and disclosed in WO2009018415 and WO2002038568. Acid 33 is transformed to the amide and dehydrated to provide nitrile 34. Palladium-catalyzed coupling of 34 with boronate 35a, which may be prepared as shown in Scheme 6, ultimately results in the preparation of biarylbromide 11f (Y¹═F). Biarylbromide 11i (Y¹═H), for use in the synthesis of compounds 109 and 111, may be prepared by the same methodology using 3-nitrophenylboronic acid (28).

Intermediate 11 j may be prepared as depicted in Scheme 5e below.

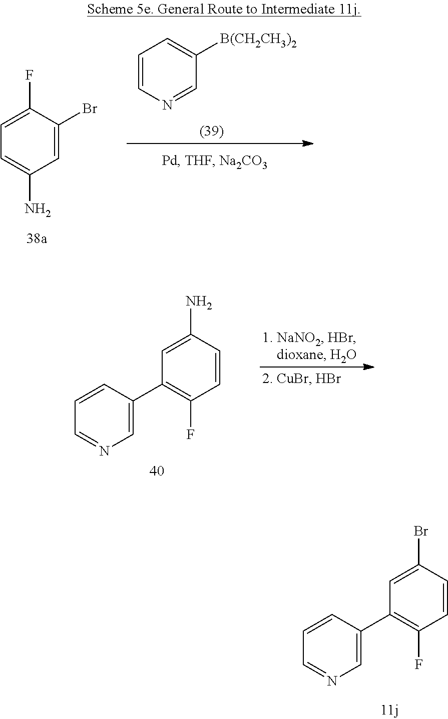

Biarylbromide 11 j, for use in the preparation of compounds 114 and 116, may be prepared from the commercially available aniline 38a according to the methods shown in Scheme 5e and disclosed in WO2003008418.

Intermediates 11k and 11m may be prepared as depicted in Scheme 5f below.

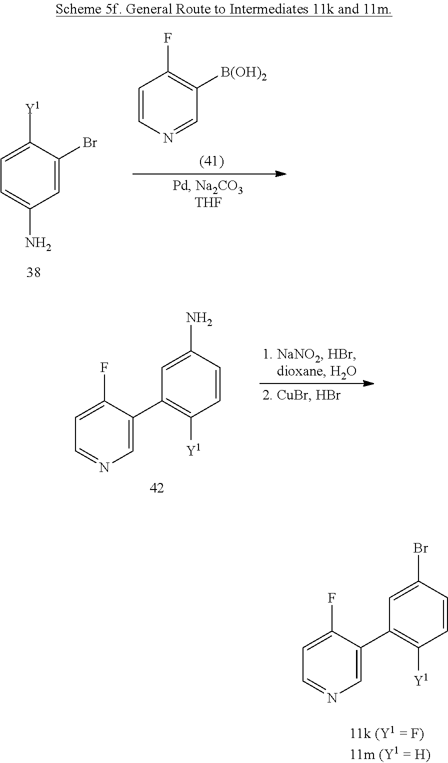

Biarylbromide 11k (Y¹═F), for use in the preparation of compounds 118 and 120, may be prepared from the commercially available aniline 38a (Y¹═F) according to the methods shown in Scheme 5f and disclosed in WO2003008418. Alternatively, starting with commercially available aniline 38b (Y¹═H), the same methodology may be used to prepare the biarylbromide 11m (Y¹═H), for use in the synthesis of compounds 117 and 119.

Intermediates 11n and 11p may be prepared as depicted in Scheme 51 below.

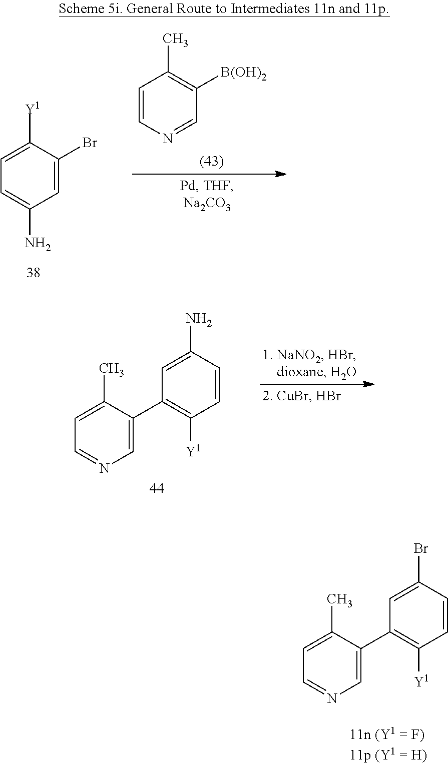

Biarylbromide 11n (Y¹═F), for use in the preparation of compounds 122 and 124, may be prepared from the commercially available aniline 38a (Y¹═F) according to the methods shown in Scheme 51 and disclosed in WO2003008418. The preparation of biarylbromide 11p (Y¹═H), for use in the synthesis of compounds 121 and 123, may be carried out using this same methodology and starting with commercially available aniline 38b (Y¹═H).

Intermediates 11q and 11r may be prepared as depicted in Scheme 5j below.

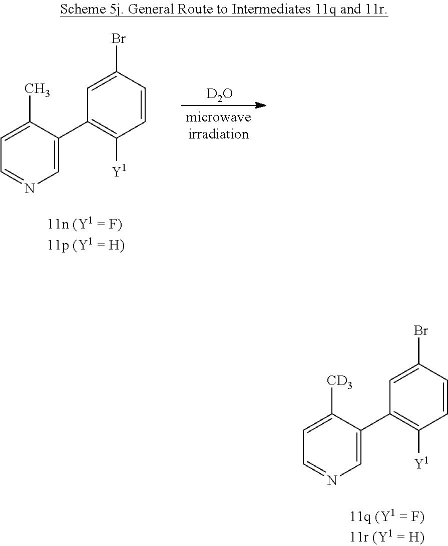

Biarylbromides 11q (Y¹═F) and 11r (Y¹═H), for use in the preparation of compounds 125 to 128, may be prepared as outlined in Scheme 5j according to the procedure described by Anto, S et al., Synthesis and Applications of Isotopically Labelled Compounds, Proc. of the 7^{th} Intl. Symp., Dresden, Germany, 2000, ed. U. Pleiss and R. Voges, Wiley, N Y, 2001, pp. 93-96, in which selective deuteration of p-methylpyridine is carried out in the presence of D₂O with microwave irradiation.

Intermediate 35a may be prepared as depicted in Scheme 6 below.

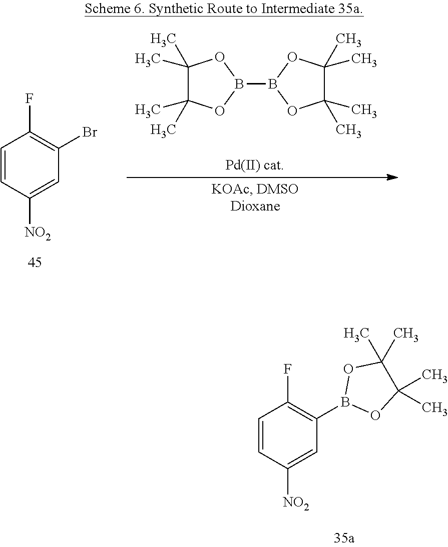

Boronate 35a, for use in Scheme 5d, may be prepared from commercially available 3-bromo-4-fluoro-nitrobenzene (45) as depicted in Scheme 6 and described in WO2003008418.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art. Combinations of substituents and variables envisioned by this invention are only those that result in the formation of stable compounds.

### Compositions

The invention also provides pyrogen-free compositions comprising an effective amount of a compound of Formula I (e.g., including any of the formulae herein), or a pharmaceutically acceptable salt of said compound; and an acceptable carrier. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, 10n exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz, J D and Zaffaroni, A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound that either antagonizes the al subunit of GABA_{A} receptors, or which is a partial agonist of the α2, α3 and/or α5 subunits of GABA_{A} receptors.

Preferably, the second therapeutic agent is an agent useful in the treatment or prevention of a disease or condition selected from anxiety disorders such as panic disorder with or without agoraphobia, agoraphobia without history of panic disorder, animal and other phobias including social phobias, obsessive-compulsive disorder, stress disorders incuding post-traumatic and acute stress disorder, and generalized or substance-induced anxiety disorder; convulsions; depressive or bipolar disorders, for example single-episode or recurrent major depressive disorder, dysthymic disorder, bipolar I and bipolar II manic disorders, and cyclothymic disorder; psychotic disorders including schizophrenia; neurodegeneration arising from cerebral ischemia; attention deficit hyperactivity disorder; speech disorders, including stuttering; and disorders of circadian rhythm, for example in subjects suffering from the effects of jet lag or shift work; emesis, including acute, delayed and anticipatory emesis, in particular emesis induced by chemotherapy or radiation, as well as motion sickness, and post-operative nausea and vomiting; eating disorders including anorexia nervosa and bulimia nervosa; premenstrual syndrome; muscle spasm or spasticity, for example in paraplegic patients; hearing disorders, including tinnitus and age-related hearing impairment; urinary incontinence; and the effects of substance abuse and dependency, including alcohol withdrawal; athetosis, epilepsy, stiff-person syndrome, pain and nociception; and other disorders of the central nervous system.

In a particular embodiment, the second therapeutic is an agent useful in the treatment or prevention of a disease or condition selected from anxiety, convulsions, skeletal muscle spasm, spasticity, athetosis, epilepsy, stiff-person syndrome, other disorders of the central nervous system, and pain (e.g., neuropathic pain, inflammatory pain, and migraine-associated pain). In another embodiment, the second therapeutic is an agent useful in the treatment or prevention of a disease or condition selected from anxiety and convulsions.

Examples of pain include acute, chronic, neuropathic, or inflammatory pain, arthritis, migraine, cluster headaches, trigeminal neuralgia, herpetic neuralgia, general neuralgias, visceral pain, osteoarthritis pain, postherpetic neuralgia, diabetic neuropathy, radicular pain, sciatica, back pain, head or neck pain, severe or intractable pain, nociceptive pain, breakthrough pain, postsurgical pain, and cancer pain. More particular examples include femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, including abdominal, pancreatic, and IBS pain; chronic and acute headache pain; migraine; tension headache, including cluster headaches; chronic and acute neuropathic pain, including post-herpetic neuralgia; diabetic neuropathy; HIV-associated neuropathy; trigeminal neuralgia; Charcot-Marie Tooth neuropathy; hereditary sensory neuropathies; peripheral nerve injury; painful neuromas; ectopic proximal and distal discharges; radiculopathy; chemotherapy induced neuropathic pain; radiotherapy-induced neuropathic pain; post-mastectomy pain; central pain; spinal cord injury pain; post-stroke pain; thalamic pain; complex regional pain syndrome; phantom pain; intractable pain; acute pain, acute post-operative pain; acute musculoskeletal pain; joint pain; mechanical low back pain; neck pain; tendonitis; injury/exercise pain; acute visceral pain, including abdominal pain, pyelonephritis, appendicitis, cholecystitis, intestinal obstruction, and hernias; chest pain, including cardiac pain; pelvic pain; renal colic pain; acute obstetric pain, including labor pain; cesarean section pain; acute inflammatory, burn and trauma pain; acute intermittent pain, including endometriosis; acute herpes zoster pain; sickle cell anemia; acute pancreatitis; breakthrough pain; orofacial pain including sinusitis pain and dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; painful bladder syndrome; interstitial cystitis (IC); prostatitis; complex regional pain syndrome (CRPS), type I and type II; and angina-induced pain. For example, the pain may be pain selected from the group consisting of fibromyalgia, acute herpes zoster pain, HIV-associated neuropathy, neuropathic low back pain, chemotherapy induced neuropathic pain, radiotherapy-induced neuropathic pain, peripheral nerve injury, spinal cord injury pain, and multiple sclerosis (MS) pain.

In one embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat the target disease or condition.

The interrelationship of dosages for animals and humans (based on milligrams per meter squared of body surface) is described in Freireich et al., (1966) Cancer Chemother. Rep 50: 219. Body surface area may be approximately determined from height and weight of the subject. See, e.g., Scientific Tables, Geigy Pharmaceuticals, Ardsley, N.Y., 1970, 537.

In one embodiment, an effective amount of a compound of this invention can range from about 0.01 to about 5000 mg per treatment. In more specific embodiments the range is from about 0.1 to 2500 mg, or from 0.2 to 1000 mg, or most specifically from about 1 to 500 mg. Treatment typically is administered one to three times daily.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

According to another embodiment, the invention provides a method of treating a disease that is beneficially treated by a compound showing partial-agonist activity at the GABA α2, α3 and α5 subtype receptors, and antagonist activity at the al subtype receptor, in a subject comprising the step of administering to said subject an effective amount of a compound of this invention or a pharmaceutically acceptable salt of said compound or a composition of this invention. Such diseases are well known in the art and are disclosed in, but not limited to the following patents and published applications: WO 1998004559, WO 2000044752, WO 2006061428 and U.S. Pat. No. 6,630,471. Such diseases include, but are not limited to, anxiety disorders such as panic disorder with or without agoraphobia, agoraphobia without history of panic disorder, animal and other phobias including social phobias, obsessive-compulsive disorder, stress disorders incuding post-traumatic and acute stress disorder, and generalized or substance-induced anxiety disorder; convulsions; depressive or bipolar disorders, for example singe-episode or recurrent major depressive disorder, dysthymic disorder, bipolar I and bipolar II manic disorders, and cyclothymic disorder; psychotic disorders including schizophrenia; neurodegeneration arising from cerebral ischemia; attention deficit hyperactivity disorder; speech disorders, including stuttering; and disorders of circadian rhythm, for example in subjects suffering from the effects of jet lag or shift work; emesis, including acute, delayed and anticipatory emesis, in particular emesis induced by chemotherapy or radiation, as well as motion sickness, and post-operative nausea and vomiting; eating disorders including anorexia nervosa and bulimia nervosa; premenstrual syndrome; muscle spasm or spasticity, for example in paraplegic patients; hearing disorders, including tinnitus and age-related hearing impairment; urinary incontinence; and the effects of substance abuse and dependency, including alcohol withdrawal; athetosis, epilepsy, stiff-person syndrome, pain and nociception; and other disorders of the central nervous system.

In a particular embodiment, the disease or conditions is selected from anxiety, convulsions, skeletal muscle spasm, spasticity, athetosis, epilepsy, stiff-person syndrome, other disorders of the central nervous system, and pain (e.g., neuropathic pain, inflammatory pain, and migraine-associated pain). In a particular embodiment, the disease is selected from anxiety and convulsions.

In one embodiment, the disease is pain selected from the group consisting of: acute, chronic, neuropathic, or inflammatory pain, arthritis, migraine, cluster headaches, trigeminal neuralgia, herpetic neuralgia, general neuralgias, visceral pain, osteoarthritis pain, postherpetic neuralgia, diabetic neuropathy, radicular pain, sciatica, back pain, head pain, neck pain, severe or intractable pain, nociceptive pain, breakthrough pain, postsurgical pain, and cancer pain.

In another embodiment, the pain is selected from the group consisting of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, including abdominal, pancreatic, and IBS pain; chronic and acute headache pain; migraine; tension headache, including cluster headaches; chronic and acute neuropathic pain, including post-herpetic neuralgia; diabetic neuropathy; HIV-associated neuropathy; trigeminal neuralgia; Charcot-Marie Tooth neuropathy; hereditary sensory neuropathies; peripheral nerve injury; painful neuromas; ectopic proximal and distal discharges; radiculopathy; chemotherapy induced neuropathic pain; radiotherapy-induced neuropathic pain; post-mastectomy pain; central pain; spinal cord injury pain; post-stroke pain; thalamic pain; complex regional pain syndrome; phantom pain; intractable pain; acute pain, acute post-operative pain; acute musculoskeletal pain; joint pain; mechanical low back pain; neck pain; tendonitis; injury/exercise pain; acute visceral pain, including abdominal pain, pyelonephritis, appendicitis, cholecystitis, intestinal obstruction, and hernias; chest pain, including cardiac pain; pelvic pain; renal colic pain; acute obstetric pain, including labor pain; cesarean section pain; acute inflammatory, burn and trauma pain; acute intermittent pain, including endometriosis; acute herpes zoster pain; sickle cell anemia; acute pancreatitis; breakthrough pain; orofacial pain including sinusitis pain and dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; painful bladder syndrome; interstitial cystitis (IC); prostatitis; complex regional pain syndrome (CRPS), type I and type II; and angina-induced pain.

In yet another embodiment, the pain is selected from the group consisting of: fibromyalgia, acute herpes zoster pain, HIV-associated neuropathy, neuropathic low back pain, chemotherapy induced neuropathic pain, radiotherapy-induced neuropathic pain, peripheral nerve injury, spinal cord injury pain, and multiple sclerosis (MS) pain.

Methods delineated herein also include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In another embodiment, any of the above methods of treatment comprises the further step of co-administering to said subject one or more second therapeutic agents. The choice of second therapeutic agent may be made from any second therapeutic agent known to be useful for co-administration with a compound that either antagonizes the al subunit of GABA_{A} receptors, or which is a partial agonist of the α2 and/or α3 subunits of GABA_{A} receptors. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

The term subject can include a patient in need of treatment.

In yet another aspect, the invention provides the use of a compound of Formula I, or a pharmaceutically acceptable salt of said compound, alone or together with one or more of the above-described second therapeutic agents in the manufacture of a medicament, either as a single composition or as separate dosage forms, for treatment or prevention in a subject of a disease, disorder or symptom set forth above. Another aspect of the invention is a compound of Formula I for use in the treatment or prevention in a subject of a disease, disorder or symptom thereof delineated herein.

### EXAMPLES

### Example 1

### Synthesis of 5′-(3-tert-butylimidazo[1,2-b][1,2,4]triazin-7-yl)-2′,6-difluorobiphenyl-2-carbonitrile (Compound 130)

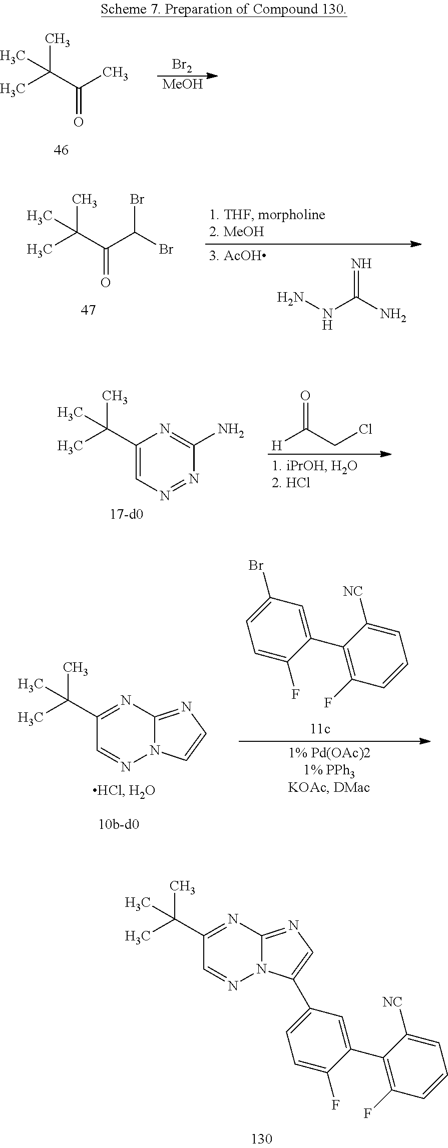

Step 1. 1,1-Dibromo-3,3-dimethylbutan-2-one (47): To a solution of 46 (5.0 g, 49.9 mmol) in methanol (50 mL) was added dropwise bromine (5.1 mL, 99.8 mmol). The reaction was stirred at room temperature overnight. NMR analysis showed approximately 20% mono-brominated material remained and the reaction was stirred for a further 5-6 hr. The reaction was quenched by the addition of saturated Na₂S₂O₃ solution (20 mL) and was diluted with H₂O until the yellow color disappeared and a white suspension was observed. The solids were isolated by filtration and dried under vacuum to provide 9.63 g (75%) of 47 as a white fluffy solid.

Step 2. 5-(tert-Butyl)-1,2,4-triazin-3-amine (17-d0): Aminoguanidine bicarbonate (4.75 g, 34.9 mmol) was suspended in methanol (25 mL). Acetic acid (6 mL, 104.7 mmol) was added dropwise and the solution was stirred at room temperature overnight.

In a separate flask 47 (9.00 g, 34.9 mmol) was dissolved in THF (50 mL). The solution was heated to 45° C. and morpholine (12.2 mL, 139.5 mmol) was added dropwise. The reaction was heated at 65-70° C. and stirred overnight. An evaporated aliquot was checked by NMR to ensure complete consumption of starting material. The reaction was cooled and the solids were removed by filtration and washed with THF. The filtrate was concentrated, then twice dissolved in methanol (2×50 mL) and reconcentrated. Finally, the material was dissolved in fresh methanol (50 mL). The previously prepared MeOH solution of aminoguanidine acetate was added slowly over 30 min. The reaction was stirred at room temperature over a weekend. Some starting material remained by GCMS. The reaction was heated at 45-50° C. overnight. The mixture was cooled and concentrated to one-half volume. Water (50 mL) and heptane (25 mL) were added with vigorous stirring. The layers were separated and the heptane layer was checked for product and discarded. The aqueous mixture was further concentrated to remove residual methanol, then was cooled in an ice bath. The product was isolated by filtration and washed with cold water. Drying provided 2.0 g (38%) of 17-d0 as a light yellow solid. Approximately 10% of a side product was visible by NMR. The material was carried forward to the next reaction.

Step 3. 3-(tert-Butyl)imidazo[1,2-b][1,2,4]triazine hydrochloride hydrate (10b-d0): To a solution of 17-d0 (1.8 g, 11.8 mmol) in isopropanol (15 mL) was added chloroacetaldehyde (50% in H₂O, 5.5 mL, 35.5 mmol) and the solution was heated at 85° C. (external) overnight. The dark suspension was cooled and diluted with heptane. Filtration gave 1.06 g (50%) of 10b-d0 hydrochloride salt hydrate as a light brown solid.

Step 4. 5′-(3-tert-Butyl)imidazo[1,2-b][1,2,4]triazin-7-yl)-2′,6-difluoro-[1,1′-biphenyl]-2-carbonitrile (Compound 130): Compound 10b-d0 hydrochloride salt hydrate (344 mg, 1.49 mmol), 11c (400 mg, 1.36 mmol, prepared as described in Example 2), potassium acetate (333 mg, 3.4 mmol) and triphenylphosphine (7.3 mg, 0.028 mmol) were added to N,N-dimethylacetamide (10 mL) and the mixture was purged with N₂ for 5 min. Palladium acetate (6 mg, 0.028 mmol) was added and the solution was heated at 130° C. (external) for 3-4 hr in a heating block. The reaction was cooled, diluted with EtOAc, and filtered through a Celite pad, washing with EtOAc until no yellow colored material remained on the pad. The organic solution was washed with brine (3×10 mL) and filtered to remove some insoluble material. All solids were discarded. The organic solution was concentrated to a residue, diluted with H₂O (50 mL), and the solids were collected by filtration. The dried solid material was combined with impure product from another 0.37-mmol-scale reaction and further purified on an Analogix automated chromatography system (12 g column, 1-30% EtOAc/heptane) to give 397 mg (60%) of 130 as a bright yellow solid. ¹H-NMR (300 MHz, CDCl₃): δ 1.49, (s, 9H), 7.35-7.41 (m, 1H), 7.47 (app dt, J=1.4, 8.4, 1H), 7.55 (app dt, J=5.3, 7.5, 1H), 7.65 (ddd, J=0.5, 1.4, 7.6, 1H), 8.15 (m, 2H), 8.22 (s, 1H), 8.58 (s, 1H). ¹³C-NMR (75 MHz, CDCl₃): δ 29.16, 37.55, 116.58, 116.67, 116.72, 116.88, 119.80, 120.02, 120.57, 120.87, 124.63, 124.78, 129.24, 129.29, 129.51, 126.56, 129.62, 130.64, 130.76, 133.72, 136.45, 157.63, 158.20, 160.98, 161.53, 161.60. HPLC (method: Waters Atlantis T3 50 mm—gradient method 5-95% ACN+0.1% formic acid in 14 min with 4 min hold at 95% can +0.1% formic acid; wavelength: 305 nm): retention time: 8.55 min; 99.8% purity. MS (M+H): 390.0. Elemental Analysis (C₂₂H₁₇F₂N₅): Calculated: C=67.86, H=4.40, F=9.76, N=17.98. Found: C=67.38, H=4.22, F=10.11, N=17.98.

### Example 2

### Synthesis of 5′-Bromo-2′,6-difluoro-[1,1′-biphenyl]-2-carbonitrile (11e)

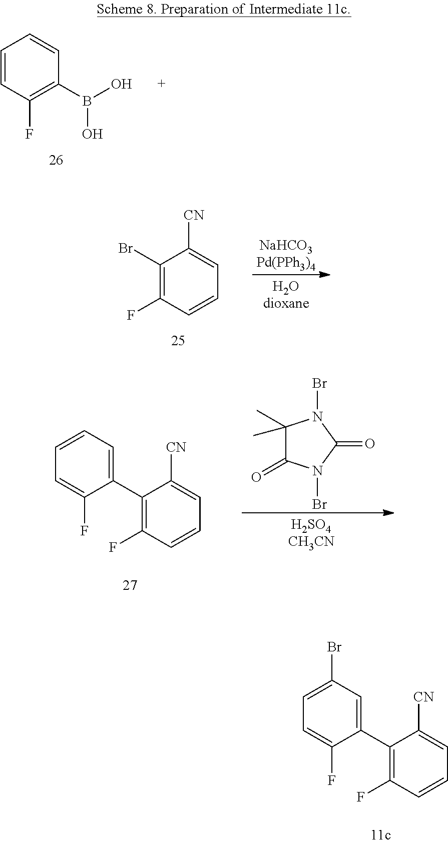

Step 1. 2′,6-Difluoro-[1,1′-biphenyl]-2-carbonitrile (27): A mixture of (2-fluorophenyl)boronic acid 26 (6.7 g, 48.0 mmol), 2-bromo-3-fluorobenzonitrile 25 (8 g, 40.0 mmol) and NaHCO₃ (8.4 g, 100 mmol) in dioxane (100 mL) and water (20 mL) was purged with N₂ for 5 min. Tetrakis(triphenylphosphine)palladium (3.2 g, 2.8 mmol) was added and the mixture heated at 80-85° C. (external) overnight. The reaction showed progress but considerable starting material remained. The reaction was heated at 95° C. (external) for a further 30 hr. The solution was cooled and diluted with EtOAc (500 mL). The mixture was washed with brine (100 mL) and the aqueous layer was back-extracted with EtOAc (2×100 mL). The combined organic solution was dried (Na₂SO₄) and concentrated to a yellow liquid. The crude material was purified by chromatography on an Analogix automated system (115 g column, 0-30% EtOAc/heptane) to provide 3.5 g (78%) of clean 27 plus 4 g of oil/solid mixture that was approximately 30% starting material by GCMS.

Step 2. 5′-Bromo-2′,6-difluoro-[1,1′-biphenyl]-2-carbonitrile (110: 1,3-dibromo-5,5-dimethylhydantoin (4.25 g, 14.9 mmol) was added to solution of 27 (3.2 g, 14.9 mmol) in acetonitrile (30 mL). Concentrated sulfuric acid (1.24 mL, 22.3 mmol) was then added dropwise. The reaction was heated at 40-45° C. for several hours then allowed to stir at room temperature overnight. Progress was checked via LCMS and the reaction was heated again until no further progress was apparent. The solution was cooled and slowly diluted with water (40 mL). The product was extracted with EtOAc to give 5.34 g of crude material. The crude material was combined with another 5.3 g of less pure material and purified on an Analogix automated chromatography system (110 g column, 0-30% EtOAc/heptane) to provide 6.4 g (81%) of 11c. NMR and LCMS showed 10-15% starting material remained. The material could be carried forward successfully to the next reaction; however, using further purified material gave higher yields in the ensuing reaction.

### Example 3

### Synthesis of 5′-(3-d9-tert-butylimidazo[1,2-b][1,2,4]triazin-7-yl)-2′,6-difluorobiphenyl-2-carbonitrile (Compound 102)

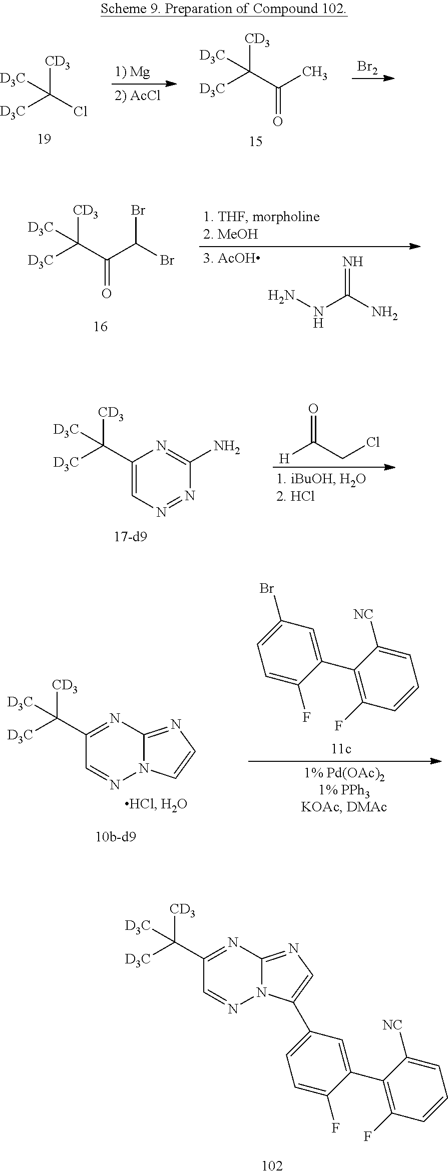

Step 1. 4,4,4-d₃-3,3-Bis(methyl-d₃)butan-2-one (15): A four-neck round bottom flask equipped with a mechanical stirrer, thermowell, nitrogen inlet and dropping funnel was charged with diethyl ether (10 mL), magnesium powder (2.36 g, 98 mmol) and a small crystal of iodine. Approximately 10 mL of a solution of t-butyl chloride-d9 (10 g, 98 mmol; Cambridge Isotopes, 98 atom % D) in diethyl ether (100 mL) was added. The solution was warmed slightly as needed to help initiate the reaction. The t-butyl chloride-d9 solution was then added at such a rate as to maintain reflux. After the addition the reaction was maintained at reflux for a further 2 hr until most of the magnesium had been consumed. The thick grey suspension was cooled to room temperature. The cooled mixture was slowly cannulated into cold (−5-0° C.) neat acetyl chloride (21 mL, 295 mmol) at such a rate as to maintain the temperature at less than 5° C. The greenish-yellow suspension was allowed to warm to room temperature and was stirred overnight. The mixture was cooled to 0° C. and 4M HCl (60 mL) was added slowly to quench the reaction. The mixture was saturated with solid NaCl. Three layers were observed and separated. The lower (mostly brine) and middle (mostly AcOH) layers were washed with diethyl ether. The combined organic solutions were stirred over solid NaHCO₃ with small amounts (1 mL) of water periodically added until an NMR of an aliquot showed most of the acetic acid had been consumed. The solution was dried (Na₂SO₄), filtered, and concentrated using a cool (less than 30° C.) water bath to afford 3.8 g (36%) of 15. The material was carried forward to the next step.

Step 2. 1,1-Dibromo-4,4,4-d₃-3,3-bis(methyl-d₃)butan-2-one (16): To a solution of 15 (estimated 70% product by NMR; 15 g) in methanol (150 mL) was added bromine (10 mL, 0.19 mol) dropwise and the reaction was stirred at room temperature over a weekend. LCMS analysis showed approximately 10% mono-brominated material and the reaction was stirred for another 48 hr until most of the mono-brominated material was consumed. The reaction was concentrated (cool 30° C. water bath) then diluted with MTBE (150 mL). The organic solution was washed with dilute NaHCO₃ solution and with brine, dried (Na₂SO₄), and concentrated (30° C. water bath) to afford 5.82 g of 16. A portion of 16 (4.1 g) was purified by dissolving in a minimum amount of dichloromethane and passing through 40 g of silica gel (3:1 heptane:EtOAc) to recover 3.75 g of pure 16 as a light yellow solid.

Step 3. 5-(tert-Butyl-d₉)-1,2,4-triazin-3-amine(17-d9): 16 (1.8 g, 6.98 mmol) was dissolved in anhydrous THF (20 mL). The solution was heated to 45° C. and morpholine (2.5 mL, 28.6 mmol) was added dropwise. The reaction was heated at 50° C. (2-3 days). An evaporated aliquot was checked by NMR to ensure complete consumption of the starting material. The reaction was cooled and the solids were removed by filtration and washed with THF. The filtrate was concentrated, then twice dissolved in methanol (2×20 mL) and reconcentrated. Finally, the material was dissolved in fresh methanol (20 mL). Aminoguanidine bicarbonate (0.95 g, 6.98 mmol) was added followed by the dropwise addition of acetic acid (1.2 mL, 21.0 mmol) over 10 min. The reaction was stirred at room temperature for 1-2 hr until bubbling ceased. The solution was then heated at 50° C. (heating block) overnight. The reaction showed some progress but starting material remained. Heating was continued at 65° C. overnight. The reaction was cooled and concentrated. Water was added and the solids removed by filtration. The product materials from a total of 2 runs (14 mmol) were combined and dried to afford 950 mg of 17-d9 as a yellow solid. A further 125 mg was obtained from the filtrate by further extraction and purification.

Step 4. 3-(tert-Butyl-d₉)imidazo[1,2-b][1,2,4]triazine hydrochloride hydrate (10b-d9): To a solution of 17-d9 (537 mg, 3.33 mmol) in isobutanol (2 mL) was added chloroacetaldehyde (50% in H₂O, 1.3 mL, 9.99 mmol) and the solution was heated at 85° C. (heating block) overnight. The dark suspension was cooled, diluted with acetonitrile, and some clean product was isolated by filtration. The filtrates were concentrated to remove volatile organics, then were washed with MTBE. The organic layer was checked for product and then discarded. The aqueous solution was made basic with 24% NaOH and re-extracted with dichloromethane until no product remained in the aqueous phase. The crude material thus recovered from multiple reaction filtrates was purified on an Analogix chromatography system (24 g column, 0-3% MeOH/dichloromethane). The material was dissolved in a small amount of acetonitrile and conc. HCl (estimated 1 equiv) was added. The product was isolated by filtration and dried. A total of 6.65 mmol of starting material provided 700 mg (44%) of 10b-d9 hydrochloride salt hydrate. MS (M+H): 186.3.

Step 5. 5′-(3-d₉-tert-Butylimidazo[1,2-b][1,2,4]triazin-7-yl)-2′,6-difluorobiphenyl-2-carbonitrile (Compound 102): Compound 10b-d9 hydrochloride salt hydrate (100 mg, 0.42 mmol), 11c (112 mg, 0.38 mmol, prepared as described in Example 2), potassium acetate (93 mg, 0.95 mmol) and triphenylphosphine (2.0 mg, 0.0076 mmol) were added to N,N-dimethylacetamide (3 mL) and the mixture was purged with N₂ for 5 min. Palladium acetate (1.7 mg, 0.0076 mmol) was added and the solution was heated at 130° C. for 3 hr. The reaction was cooled, diluted with EtOAc (50 mL) and filtered through a Celite pad, washing with EtOAc until no yellow colored material remained on the pad. The organic solution was washed with brine (2×10 mL) then concentrated. The residue was diluted with H₂O (50 mL) and the solids were collected by filtration. The solid was re-dissolved in a small amount of EtOAc, dried (Na₂SO₄), and re-concentrated. The material was combined with impure product from another 0.37-mmol-scale reaction and further purified on an Analogix automated chromatography system (12 g, column 0-30% EtOAc/heptane) to give 75 mg (26%) of 102 as a bright yellow solid. ¹H-NMR (300 MHz, CDCl₃): δ 7.35-7.59 (m, 2H), 7.65 (ddd, J=0.6, 1.4, 7.6, 1H), 8.11-8.18 (m, 2H), 8.22 (s, 1H), 8.57 (s, 1H). ¹³C-NMR (75 MHz, CDCl₃): δ 116.58, 116.88, 120.58, 120.87, 129.29, 129.62, 130.76, 133.72, 136.45. HPLC (method: Waters Atlantis T3 50 mm—gradient method 5-95% ACN+0.1% formic acid in 14 min with 4 min hold at 95% ACN+0.1% formic acid; wavelength: 305 nm): retention time: 8.52 min; 98.7% purity. MS (M+H): 399.0. Elemental Analysis (C₂₂H₈D₉F₂N₅): Calculated: C=66.32, H=4.30, F=9.54, N=17.58. Found: C=66.98, H=4.25, F=9.32, N=17.18.

### Example 4

### Synthesis of 3-(tert-butyl-d9)-7-(3-(3,5-difluoropyridin-2-yl)-4-fluorophenyl)imidazo[1,2-b][1,2,4]triazine (Compound 106)

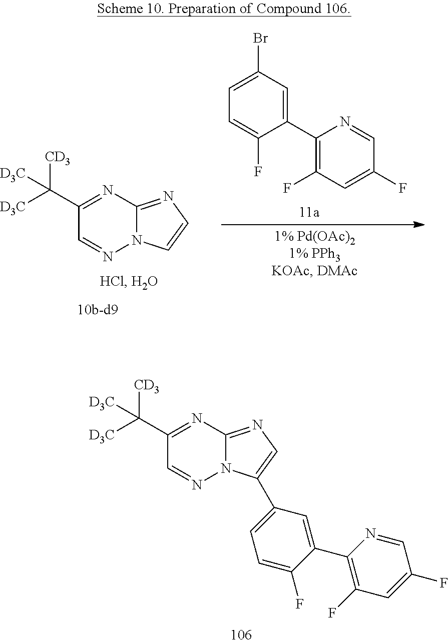

3-(tert-butyl-d9)-7-(3-(3,5-difluoropyridin-2-yl)-4-fluorophenyl)imidazo[1,2-b][1,2,4]triazine (Compound 106): Compound 10b-d9 hydrochloride salt hydrate (146 mg, 0.61 mmol, prepared as described in Example 3), 11a (200 mg, 0.56 mmol, prepared as described in Example 5), potassium acetate (137 mg, 1.4 mmol) and triphenylphosphine (2.9 mg, 0.011 mmol) were added to N,N-dimethylacetamide (3 mL) and the mixture was purged with N₂ for 5 min. Palladium acetate (2.5 mg, 0.011 mmol) was added and the solution was heated at 130° C. for 2 hr. The reaction was cooled, diluted with EtOAc (30 mL) and filtered through a Celite pad, washing with EtOAc until no yellow colored material remained on the pad. The filtrate was washed with brine (10 mL), dried (Na₂SO₄) and concentrated. The crude product was purified twice on an Analogix automated system (24 g column, 0-50% EtOAc/heptane) to provide 80 mg of 106 as a bright yellow solid. ¹H-NMR (300 MHz, CDCl₃): δ 7.26-7.40 (m, 2H), 8.14 (ddd, J=2.4, 4.8, 8.7, 1H), 8.22 (s, 1H), 8.27 (dd, J=2.3, 6.8, 1H), 8.52 (d, J=2.5, 1H), 8.57 (s, 1H). ¹³C-NMR (75 MHz, CDCl₃): δ 111.68, 111.99, 112.26, 116.39, 116.69, 129.05, 129.17, 129.48, 133.67, 134.07, 134.13, 134.43, 136.41. HPLC (method: Waters Atlantis T3 50 mm—gradient method 5-95% ACN+0.1% formic acid in 14 min with 4 min hold at 95% ACN+0.1% formic acid; wavelength: 305 nm): retention time: 8.33 min; 99.7% purity. MS (M+H): 393.0. Elemental Analysis (C₂₀H₇D9F3N5): Calculated: C=61.22, H=4.11, F=14.53, N=17.85. Found: C=62.70, H=4.50, F=14.37, N=16.75.

### Example 5

### Synthesis of 2-(5-Bromo-2-fluorophenyl)-3,5-difluoropyridine (11a)

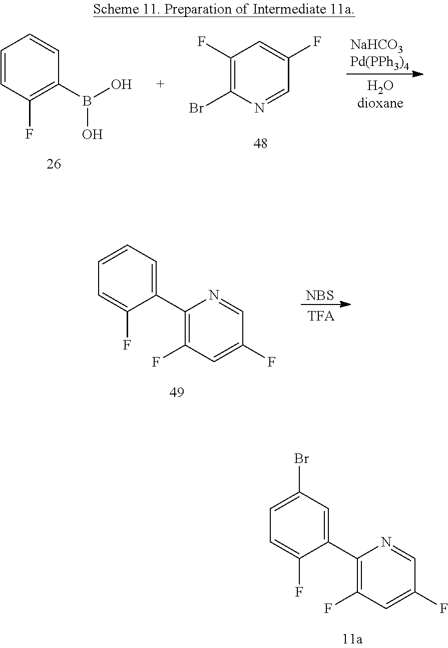

Step 1. 3,5-Difluoro-2-(2-fluorophenyl)pyridine (49): A solution of (2-fluorophenyl)boronic acid 26 (779 mg, 5.56 mmol), 2-bromo-3,5-difluoropyridine 48 (900 mg, 4.64 mmol) and sodium bicarbonate (974 mg, 11.6 mmol) in dioxane (16 mL) and water (4 mL) was purged with N₂ for 5 min.

Tetrakis(triphenylphosphine)palladium (536 mg, 0.46 mmol) was added and the mixture was heated at 80° C. (heating block) over a weekend. The solution was cooled and the volatile organic material was evaporated. The residue was partitioned between EtOAc (100 mL) and water (10 mL) and the layers were separated. The aqueous layer was back-extracted with EtOAc (20 mL). The combined organic solution was dried (Na₂SO₄) and concentrated. The crude material was combined with crude material from another 0.52-mmol-scale reaction and purified on an Analogix automated system (40 g column, 0-50% EtOAc/heptane) to provide 840 mg (78%) of 49.

Step 2. 2-(5-Bromo-2-fluorophenyl)-3,5-difluoropyridine (11a): Compound 49 (600 mg, 2.87 mmol) was dissolved in TFA (5 mL). N-Bromosuccinimide (536 mg, 3.01 mmol) was added and the solution was heated at 40-45° C. (heating block) over a weekend. The mixture was diluted with dichloromethane and washed with H₂O. The aqueous phase was made basic with 24% NaOH and back-extracted with dichloromethane. The combined organic solutions were then dried (Na₂SO₄) and concentrated. The crude material was purified on an Analogix automated system (12 g column, 0-10% EtOAc/heptane) to afford 550 mg of 11a. NMR showed some residual starting material remained, but the product was carried forward to the next reaction.

### Example 6

### Synthesis of 3-(tert-butyl-d9)-7-(3-(pyridin-3-yl)phenyl)imidazo[1,2b][1,2,4]triazine (Compound 113)

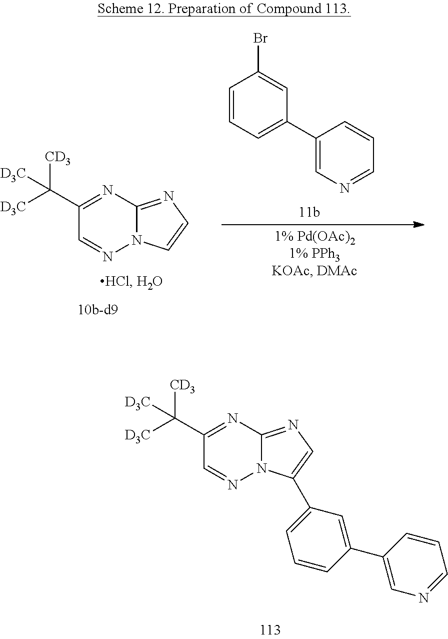

3-(tert-Butyl-d9)-7-(3-(pyridin-3-yl)phenyl)imidazo[1,2-b][1,2,4]triazine (Compound 113): Compound 10b-d9 hydrochloride salt hydrate (100 mg, 0.42 mmol, prepared as described in Example 3), 11b (93 mg, 0.40 mmol, prepared as described in Example 7), potassium acetate (100 mg, 1.0 mmol) and triphenylphosphine (2.0 mg, 0.0076 mmol) were added to N,N-dimethylacetamide (3 mL) and the mixture was purged with N₂ for 5 min. Palladium acetate (1.7 mg, 0.0076 mmol) was added and the solution was heated at 130° C. for 3 hr. The reaction was cooled, diluted with EtOAc (30 mL) and filtered through a Celite pad, washing with EtOAc until no yellow colored material remained on the pad. The filtrate was washed with brine (10 mL), dried (Na₂SO₄) and concentrated. The crude products from two runs at this scale (total 0.8 mmol) were combined and purified twice on an Analogix automated system (12 g column, then an 8 g column, EtOAc/heptane). The cleanest fractions were combined to provide 116 mg of 113. ¹H-NMR (300 MHz, CDCl₃): δ 7.42 (ddd, J=0.8, 4.9, 7.9, 1H), 7.59-7.66 (m, 2H), 7.96 (ddd, J=1.6, 2.4, 7.9, 1H), 8.01-8.05 (m, 1H), 8.27 (s, 1H), 8.30-8.32 (m, 1H), 8.59 (s, 1H), 8.65 (dd, J=1.6, 4.8, 1H), 8.94 (dd, J=0.8, 2.4, 1H). ¹³C-NMR (75 MHz, CDCl₃): δ 123.64, 125.08, 126.03, 126.94, 128.92, 129.61, 133.90, 134.48, 136.32, 136.43, 138.55, 148.48, 148.86. HPLC (method: Waters Atlantis T3 50 mm—gradient method 5-95% ACN+0.1% formic acid in 14 min with 4 min hold at 95% ACN+0.1% formic acid; wavelength: 305 nm): retention time: 5.17 min; 99.8% purity. MS (M+H): 339.3. Elemental Analysis (C₂₀H₁₀D₉N₅): Calculated: C=70.99, H=5.66, N=20.70. Found: C=70.82, H=5.78, N=20.49.

### Example 7

### Synthesis of 3-(3-bromophenyl)pyridine (11b)

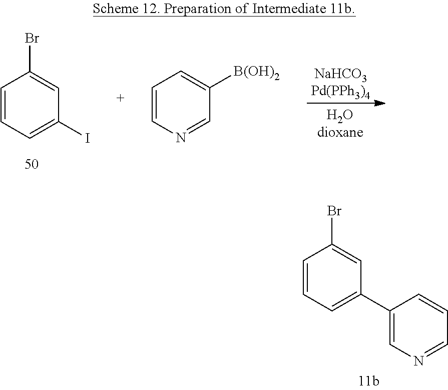

3-(3-Bromophenyl)pyridine (11b): A solution of pyridine 3-boronic acid 51 (1.0 g, 8.14 mmol), 3-bromo-1-iodobenzene 50 (2.0 g, 7.07 mmol) and sodium bicarbonate (1.5 g, 17.7 mmol) in dioxane (16 mL) and water (4 mL) was purged with N₂ for 5 min. Tetrakis(triphenylphosphine)palladium (400 mg, 0.35 mmol) was added and the mixture was heated at 90° C. for 24 hr. The solution was cooled and the volatile organics were evaporated. The residue was partitioned between EtOAc (50 mL) and water and the layers were separated. The organic layer was washed with water, dried (Na₂SO₄), and concentrated to give 2.2 g of yellow oil. The crude material was purified on an Analogix automated system (24 g column, 0-30% EtOAc/heptane) to provide 760 mg (46%) of pure 11b. Some impure material was set aside.

### Example 8

### Synthesis of 2′,6-difluoro-5′-(3-(2-hydroxy-1,1,1,3,3,3-d6-propan-2-yl)imidazo[1,2-b][1,2,4]triazin-7-yl)biphenyl-2-carbonitrile (Compound 104)

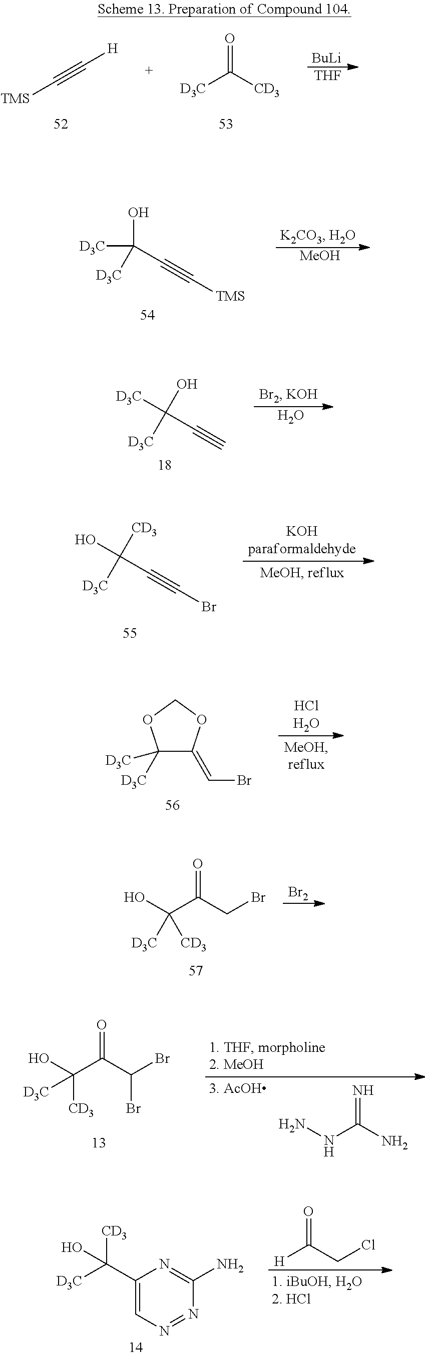

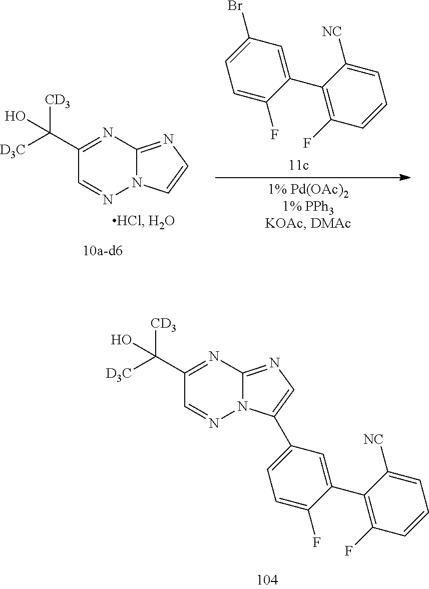

Step 1. 1,1,1-d₃-2-(Methyl-d₃)-4-(trimethylsilyl)but-3-yn-2-ol (54): A 4-neck round bottom flask equipped with a mechanical stirrer, thermowell, nitrogen inlet and dropping funnel was charged with trimethylsilyl acetylene 52 (98 mL, 0.78 mol) and THF (1 L) and the solution was cooled to 0° C. in an ice/salt bath. n-BuLi (2.5M in hexane, 312 mL, 0.78 mol) was added dropwise over 1 hr and the mixture was stirred for a further 30 min. The reaction was cooled to −78° C. and a solution of acetone-d6 53 (50 g, 0.78 mol; Cambridge Isotopes, 99.9 atom % D) in THF (50 mL) was added dropwise. The reaction was stirred for 15 min then warmed to 0° C. and stirred for a further 30 min. The reaction was quenched with water (100 mL) then diluted with MTBE (200 mL). Additional water was added until all solids dissolved. The layers were separated and the aqueous phase was extracted with MTBE (2×200 mL). The combined organic solution was washed with brine, dried (Na₂SO₄) and concentrated using a cool (25° C.) bath to give 163 g of 54. NMR indicated residual solvents remained. Material was carried forward to the next step.

Step 2. 1,1,1-d₃-2-(Methyl-d₃)but-3-yn-2-ol (18): To a solution of 54 (estimated 0.60 mol) in methanol (500 mL) and water (20 mL) was added powdered potassium carbonate (40 g, 0.30 mol). The reaction mixture was stirred at room temperature for 3-4 hr monitoring by TLC (3:1 heptane:EtOAc, KMnO₄ stain) or GCMS (in-house method SP20LOWMS, see below). The reaction was filtered, the solids were washed with a small amount of MeOH, and the filtrate was concentrated using a cool (20° C.) bath until 130 g of material remained. At this point product could be seen starting to distill over in the rotovap. The material remaining was carried forward without further concentration.

GCMS SP20LOWMS method: 30° C. for 2 min; then ramp 5° C./min to 85° C.; then ramp 20° C./min to 250° C. Column; Agilent HP-5MS 5% phenyl methyl siloxane capillary column 30.0 m×250 μm×0.25 μm.

Step 3. 4-Bromo-1,1,1-d₃-2-(methyl-d₃)but-3-yn-2-ol (55): To a cold (−5-0° C.) solution of KOH (235 g, 4.2 mol) in water (800 mL) was added dropwise bromine (30.8 mL, 0.6 mol) keeping the temperature less than 0° C. The solution was stirred for 15 min then the solution of 18 (0.6 mol) from above was added dropwise keeping the temperature at 0-5° C. The yellow color dissipated after approximately half the material was added. The solution was stirred a further 30 min, then was warmed to room temperature and checked for completion by TLC (3:1 heptane:EtOAc, KMnO₄ stain). The reaction was diluted with MTBE (500 mL) and the layers were separated. The aqueous phase was further extracted with MTBE (3×100 mL). The combined organic solution was dried (Na₂SO₄) and concentrated using a cool (20-25° C.) bath to give 140 g of material. The material was stored cold until use and was carried forward crude. NMR showed MTBE and some residual starting material were present.

Step 4. 5-(Bromomethylene)-4,4-bis(methyl-d₃)-1,3-dioxolane (56): A solution of 55 (estimated 0.57 mol), paraformaldehyde (60 g) and KOH (30 g) in MeOH (350 mL) was heated to reflux and monitored by TLC/GCMS. The reaction appeared to be complete after 3-4 hr. The solution was cooled and concentrated to half volume using a cool (20-25° C.) bath. The residue was saturated with solid NaCl and the product extracted with dichloromethane (300 mL). The organic layer was washed with brine (2×100 mL). The combined aqueous solution was then back-extracted with dichloromethane (2×100 mL). The combined organic solution was dried (Na₂SO₄) and concentrated to afford 110 g of yellow liquid. NMR showed some solvents remained. The material was purified on an Analogix automated system (400 g column, 5-30% EtOAc/hexane). Concentration using a cool (20-25° C.) bath gave 36 g of 56 (approx 32% for 3 steps) with trace solvent remaining

Step 5. 1-Bromo-4,4,4-d₃-3-hydroxy-3-(methyl-d₃)butan-2-one (57): Crude compound 56 (21 g) was dissolved in methanol (100 mL) and water (20 mL). Concentrated HCl (4 mL) was added and the mixture was heated to reflux monitoring by GCMS and NMR. After 2-3 hr starting material appeared to be mostly consumed. The reaction was heated a further 1 hr then cooled, and the methanol was evaporated using a cool (30° C.) bath. The residue was neutralized with solid NaHCO₃ and the product was extracted with MTBE. The organic solution was dried (Na₂SO₄) and concentrated over a cool bath until 13.2 g of 57 remained as a light yellow liquid. NMR showed some unknown impurity peaks and residual solvent. The material was carried forward to the next step.

Step 6. 1,1-Dibromo-4,4,4-d₃-3-hydroxy-3-(methyl-d₃)butan-2-one (13): To a solution of 57 (13 g, 69.5 mmol) in methanol (100 mL) was added bromine (3.6 mL, 69.5 mmol) dropwise and the reaction was stirred at room temperature over a weekend. Considerable mono-brominated material remained and the reaction was left for a further 24 hr. The mixture was concentrated to about half volume (cool 30° C. bath) then poured into saturated Na₂S₂O₃ solution (100 mL). The aqueous mixture was saturated with solid NaCl and the product was extracted with dichloromethane (3×50 mL). The combined organic solution was dried (Na₂SO₄) and concentrated using a cool (30° C.) bath to yield 12.8 g of material. NMR showed impurity peaks and residual solvent. The material was dissolved in a minimum amount of dichloromethane and passed through an Analogix automated chromatography system (40 g column, 20% EtOAc/heptane) to afford 8.3 g of 13 as a light yellow liquid.

Step 7. 2-(3-Amino-1,2,4-triazin-5-yl)-1,1,1,3,3,3-d6-propan-2-ol (14): Compound 13 (8.2 g, 30.7 mmol) was dissolved in anhydrous THF (50 mL). Morpholine (11 mL, 126 mmol) was added and the reaction was heated at 65-67° C. overnight. An evaporated aliquot was checked by NMR to ensure complete consumption of starting material. The reaction was cooled and the solids were removed by filtration and washed with THF. The filtrate was concentrated, then repeatedly dissolved in methanol (3×50 mL) and reconcentrated. Finally, the material was dissolved in fresh methanol (50 mL). Aminoguanidine bicarbonate (4.18 g, 30.7 mmol) was added followed by the dropwise addition of acetic acid (5.3 mL, 92.1 mmol) over 10 min. The reaction was stirred at room temperature for 1-2 hr until bubbling ceased. The reaction was then heated at 65-67° C. (heating block) overnight. The reaction was cooled and concentrated to half volume. Water (20 mL) and heptane (20 mL) were added with vigorous stirring. The layers were separated and the heptane layer was checked for product and discarded. The aqueous mixture was further concentrated to remove residual methanol, then the product was extracted with EtOAc (3×50 mL). The aqueous layer was made slightly basic with 24% NaOH solution then extracted once more with EtOAc. The combined organic solution was washed with dilute NaHCO₃ solution, brine, dried (Na₂SO₄) and concentrated to give 3.5 g (71%) of 14 which was carried forward without further purification.

Step 8. 2-(Imidazo[1,2-b][1,2,4]triazin-3-yl)-1,1,1,3,3,3-d6-propan-2-ol hydrochloride hydrate (10a-d6): To a solution of 14 (3.45 g, 21.6 mmol) in isobutanol (17 mL) was added chloroacetaldehyde (50% in H₂O, 8.3 mL, 64.7 mmol) and the solution was heated at 85° C. (heating block) overnight. The dark reaction was cooled and diluted with EtOAc (100 mL) and water (20 mL). Solid NaHCO₃ was added until the aqueous layer was slightly basic. The layers were separated and the aqueous layer was washed with EtOAc (2×100 mL). The combined organic solution was dried (Na₂SO₄) and concentrated. Crude material (from combined reactions totalling 36.6 mmol) was purified on an Analogix chromatography system (80 g column, 0-5% MeOH/dichloromethane). The recovered product was dissolved in acetonitrile (20 mL) and conc. HCl (1.7 mL, 1 equiv) was added. The product was isolated by filtration and dried to give 3.5 g (40%) of 10a-d6 hydrochloride salt hydrate as a light beige solid. MS (M+H): 184.9.

Step 9. 2′,6-difluoro-5′-(3-(2-hydroxy-1,1,1,3,3,3-d₆-propan-2-yl)imidazo[1,2-b][1,2,4]triazin-7-yl)biphenyl-2-carbonitrile (Compound 104): Compound 10a-d₆ hydrochloride salt hydrate (500 mg, 2.1 mmol), 11e (617 mg, 2.1 mmol, prepared as described in Example 2), potassium acetate (515 mg, 5.295 mmol) and triphenylphosphine (11 mg, 0.04 mmol) were added to N,N-dimethylacetamide (10 mL) and the mixture was purged with N₂ for 5 min. Palladium acetate (9.4 mg, 0.04 mmol) was added and the solution was heated at 130° C. (heating block) for 3-4 hr. The reaction was cooled, diluted with EtOAc (50 mL) and filtered through a Celite pad, washing with EtOAc until no yellow colored material remained on the pad. The filtrate was washed with brine (10 mL) then concentrated. The crude product was purified twice on an Analogix automated system (24 g column, 0-100% EtOAc/heptane) to give 340 mg of material which contained residual DMAc. Crystallization from EtOAc/heptane gave 268 mg (32%) of 104 as a bright yellow solid. ¹H-NMR (300 MHz, CDCl₃): δ 7.39 (t, J=9.3, 1H), 7.44-7.59 (m, 2H), 7.65 (ddd, J=0.5, 1.3, 7.55, 1H), 8.12-8.17 (m, 2H), 8.26 (s, 1H), 8.78 (s, 1H). ¹³C-NMR (75 MHz, CDCl₃): δ 95.50, 116.65, 116.95, 120.59, 120.89, 124.37, 124.43, 125.27, 129.242, 129.29, 129.65, 129.77, 130.69, 130.80, 134.21, 135.88, 157.79. HPLC (method: Waters Atlantis T3 50 mm—gradient method 5-95% ACN+0.1% formic acid in 14 min with 4 min hold at 95% ACN+0.1% formic acid; wavelength: 305 nm): retention time: 6.69 min; 99.8% purity. MS (M+H): 398.1. Elemental Analysis (C₂₁H₉D₆F₂N₅O): Calculated: C=63.47, H=3.80, F=9.56, N=17.62. Found: C=63.33, H=3.92, F=9.55, N=17.46.

### Example 9

### Synthesis of 2-(7-(3-(3,5-difluoropyridin-2-yl)-4-fluorophenyl)imidazo[1,2-b][1,2,4]triazin-3-yl)-1,1,1,3,3,3-d6-propan-2-ol (Compound 108)

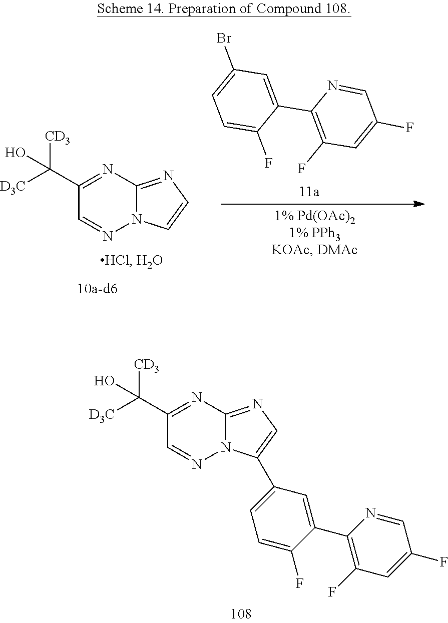

2-(7-(3-(3,5-difluoropyridin-2-yl)-4-fluorophenyl)imidazo[1,2-b][1,2,4]triazin-3-yl)-1,1,1,3,3,3-d6-propan-2-ol (Compound 108): Compound 10a-d6 hydrochloride salt hydrate (220 mg, 0.92 mmol, prepared as described in Example 8), 11a (300 mg, 0.83 mmol, prepared as described in Example 5), potassium acetate (203 mg, 2.08 mmol) and triphenylphosphine (4.5 mg, 0.017 mmol) were added to N,N-dimethylacetamide (3 mL) and the mixture was purged with N₂ for 5 min. Palladium acetate (3.7 mg, 0.017 mmol) was added and the solution was heated at 130° C. for 3 hr. The reaction was cooled, diluted with EtOAc (30 mL) and filtered through a Celite pad, washing with EtOAc until no yellow colored material remained on the pad. The filtrate was washed with brine (10 mL), dried (Na₂SO₄) and concentrated. The crude product was purified twice on an Analogix automated system (24 g, column 0-50% EtOAc/heptane) and the cleanest fractions were combined to provide 150 mg of 108. ¹H-NMR (300 MHz, CDCl₃): δ 7.31-7.40 (m, 2H), 8.14 (ddd, J=2.4, 4.9, 8.7, 1H), 8.25-8.28 (m, 2H), 8.52 (d, J=2.4, 1H), 8.77 (s, 1H). ¹³C-NMR (75 MHz, CDCl₃): δ 111.69, 111.98, 112.28, 116.45, 116.76, 124.36, 125.52, 129.19, 129.30, 129.66, 134.08, 134.16, 134.38, 134.44, 135.85, 158.56. HPLC (method: Waters Atlantis T3 50 mm—gradient method 5-95% ACN+0.1% formic acid in 14 min with 4 min hold at 95% ACN+0.1% formic acid; wavelength: 305 nm): retention time: 6.37 min; 99.8% purity. MS (M+H): 392.1. Elemental Analysis (C₁₉H₈D₆F₃N₅O): Calculated: C=58.31, H=3.61, F=14.56, N=17.90. Found: C=58.51, H=3.50, F=14.28, N=17.72.

### Example 10

### 2-(7-(3-(pyridin-3-yl)phenyl)imidazo[1,2-b][1,2,4]triazin-3-yl)1,1,1,3,3,3-d6-propan-2-ol (Compound 115)

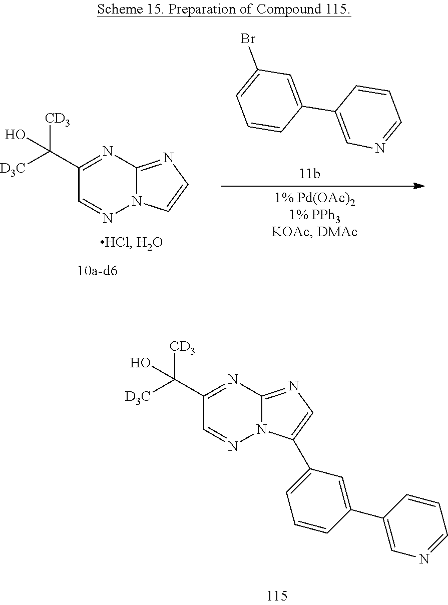

2-(7-(3-(pyridin-3-yl)phenyl)imidazo[1,2-b][1,2,4]triazin-3-yl)-1,1,1,3,3,3-d6-propan-2-ol (Compound 115: Compound 10a-d6 hydrochloride salt hydrate (500 mg, 2.1 mmol, prepared as described in Example 8), 11b (445 mg, 1.91 mmol, prepared as described in Example 7), potassium acetate (466 mg, 4.75 mmol) and triphenylphosphine (10 mg, 0.038 mmol) were added to N,N-dimethylacetamide (10 mL) and the mixture was purged with N₂ for 5 min. Palladium acetate (8.5 mg, 0.038 mmol) was added and the solution was heated at 130° C. for 3 hr. The reaction was then cooled, diluted with EtOAc (50 mL) and filtered through a Celite pad, washing with EtOAc until no yellow colored material remained on the pad. The filtrate was washed with brine (10 mL), dried (Na₂SO₄) and concentrated. The crude product was purified on an Analogix automated system (24 g, column 0-100% EtOAc/heptane) to provide 390 mg (59%) of 115 as a bright yellow solid. ¹H-NMR (300 MHz, CDCl₃): δ 7.42 (ddd, J=0.8, 4.9, 8.0, 1H), 7.63-7.65 (m, 2H), 7.96 (ddd, J=1.8, 2.3, 7.9, 1H), 8.03-8.06 (m, 1H), 8.29-8.31 (m, 1H), 8.32 (s, 1H), 8.65 (dd, J=1.6, 4.8, 1H), 8.80 (s, 1H), 8.93 (dd, J=0.9, 2.3, 1H). ¹³C-NMR (75 MHz, CDCl₃): δ 122.35, 123.88, 124.85, 125.90, 127.26, 128.34, 133.04, 133.18, 134.61, 134.92, 137.26, 147.07, 147.54. HPLC (method: Waters Atlantis T3 50 mm—gradient method 5-95% ACN+0.1% formic acid in 14 min with 4 min hold at 95% ACN+0.1% formic acid; wavelength: 305 nm): retention time: 3.48 min; 99.0% purity. MS (M+H): 338.1. Elemental Analysis (C19H11D6N50): Calculated: C=67.64, H=5.08, N=20.76. Found: C=65.99, H=4.77, N=20.00.

### Example 11

### Evaluation of Metabolic Stability in Human Liver Microsomes

Human liver microsomes (20 mg/mL) are available from Xenotech, LLC (Lenexa, Kans.). β-nicotinamide adenine dinucleotide phosphate, reduced form (NADPH), magnesium chloride (MgCl₂), and dimethyl sulfoxide (DMSO) are available from Sigma-Aldrich.

7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plat before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for TPA-023B and the positive control, 7-ethoxycoumarin (1 μM). Testing is done in triplicate.

The in vitro t_{1/2s} for test compounds are calculated from the slopes of the linear regression of % parent remaining (1n) vs incubation time relationship:

in vitro t_{1/2}=0.693/k

k=−[slope of linear regression of % parent remaining(1n) vs incubation time]. Data analysis is performed using Microsoft Excel Software.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound of Formula I: 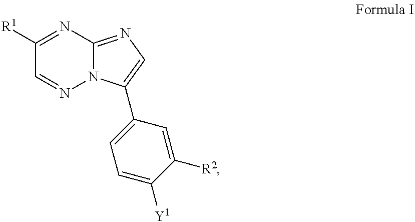
or a pharmaceutically acceptable salt thereof, wherein:
R¹ is
(a) C₁-C₆ alkyl that is optionally substituted with one or two groups selected from halogen and —OH;
(b) —OC₁-C₆ alkyl;
(c) —C(O)H;
(d) —C(O)C₁-C₆ alkyl;
(e) —C(O)OC₁-C₆ alkyl; or
(f) —CR³═NOR⁴,
wherein, R¹ is optionally substituted with one or more deuterium;
R² is aryl or heteroaryl wherein R² is optionally substituted with one or two groups independently selected from the group consisting of halogen, —OCH₃, —OCD₃, —CH₂OH, —CD₂OH, —CH₃, —CD₃, —CH₂CH₃, —CD₂CH₃, —CH₂CD₃, —CD₂CD₃, —CF₃, —CN, —C(O)H, —C(O)OCH₃, —NH₂, —C(O)CH₃, —C(O)CD₃, —SCH₃, —SCD₃, —S(O)CH₃, —S(O)CD₃, —S(O₂)CH₃, —S(O₂)CD₃, and —CH═NOH;
R³ is selected from hydrogen, deuterium, and C₁-C₆ alkyl that is optionally substituted with one or more deuterium;
R⁴ is C₁-C₆ alkyl that is optionally substituted with one or more hydroxyl or with one or more —N(C₁-C₆ alkyl)₂ wherein each alkyl in R⁴ is optionally substituted with one or more deuterium; and
Y¹ is hydrogen, —Cl, or —F;
with the proviso that at least one of R¹ and R² comprises deuterium.

2. The compound of claim 1, wherein R¹ is selected from methyl, fluoromethyl, difluoromethyl, hydroxymethyl, hydroxyethyl, difluoroethyl, fluoropropyl, hydroxypropyl, t-butyl, O-methyl, —C(O)H, —C(O)methyl, -carbonyloxymethyl and —CR³═NOR⁴, wherein R¹ is optionally substituted with one or more deuterium.

3. The compound of claim 2, wherein R¹ is selected from —CH₃, —CD₃, —CF₂CH₃, —CF₂CD₃, —CF(CH₃)₂, —CF(CD₃)₂, —C(OH)(CH₃)₂, —C(OH)(CD₃)₂, —C(CH₃)₃, and —C(CD₃)₃.

4. The compound of claim 1, 2 or 3, wherein R² is selected from the group consisting of phenyl, pyridazinyl, pyrimidinyl, pyrazinyl, furyl, pyrrolyl, pyrazolyl, oxazolyl, isoxazolyl, imidazolyl, oxadiazolyl, thiadiazolyl, triazolyl, tetrazolyl, pyridyl, thienyl, and thiazolyl wherein R² is optionally substituted with one or two groups independently selected from the group consisting of —F, —OCH₃, —OCD₃, —CH₂OH, —CD₂OH, —CH₃, —CD₃, —CH₂CH₃, —CD₂CH₃, —CH₂CD₃, —CD₂CD₃, —Cl, —CF₃, —CN, —C(O)H, —C(O)OCH₃, —NH₂, —C(O)CH₃, —C(O)CD₃, —SCH₃, —SCD₃, —S(O)CH₃, —S(O)CD₃, and —CH═NOH.

5. The compound of claim 4, wherein R² is phenyl, pyridyl, thienyl, or thiazolyl, wherein R² is optionally substituted with one or two groups selected from —F, —OCH₃, —OCD₃, —CH₃, —CD₃, —CH₂CH₃, —CD₂CH₃, —CH₂CD₃, —CD₂CD₃, —CF₃, —CN, and —C(O)H; R³ is selected from hydrogen, deuterium, —CH₃, and —CD₃; and R⁴ is selected from methyl, ethyl, hydroxyethyl, and dimethylaminoethyl, wherein each alkyl in R⁴ is optionally substituted with one or more deuterium.

6. The compound of claim 1, wherein Y¹ is hydrogen or —F.

7. The compound of claim 1, wherein the compound is a compound of Formula Ia: 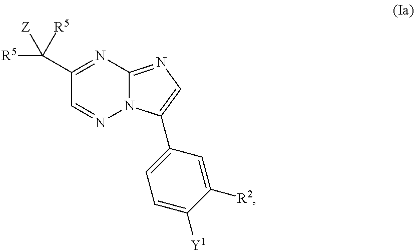
or a pharmaceutically acceptable salt thereof, wherein:
Z is —OH or methyl, wherein the methyl of Z is optionally substituted with one or more deuterium;
each R⁵ is methyl, wherein each R⁵ is optionally independently substituted with one or more deuterium; and
Y¹ is hydrogen or —F;
with the proviso that if each R⁵ is not substituted with deuterium; and Z is not substituted with deuterium; then R² comprises deuterium.

8. The compound of claim 7, wherein —CZ(R⁵)₂ is —C(CH₃)₃, —C(CD₃)₃, —C(OH)(CH₃)₂, or —C(OH)(CD₃)₂.

9. The compound of claim 8, wherein —CZ(R⁵)₂ is —C(CD₃)₃ or —C(OH)(CD₃)₂.

10. The compound of claim 7, wherein R² is phenyl or pyridyl, wherein R² is optionally substituted as defined in claim 7.

11. The compound of claim 7, wherein Y¹ is hydrogen.

12. The compound of claim 7, wherein Y¹ is —F.

13. The compound of claim 11 wherein —CZ(R⁵)₂ is —C(CD₃)₃.

14. The compound of claim 11 wherein —CZ(R⁵)₂ is —C(OH)(CD₃)₂.

15. The compound of claim 11, wherein R² is phenyl optionally substituted with one or two groups independently selected from —CH₃, —CD₃, —CN, and —F.

16. The compound of claim 15 wherein R² is phenyl optionally substituted with one or two groups independently selected from —CN, and —F.

17. The compound of claim 16 wherein R² is

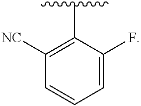

18. The compound of claim 11, wherein R² is pyridyl optionally substituted with one or two groups independently selected from —CH₃, —CD₃, —CN, and —F.

19. The compound of claim 18, wherein the pyridyl nitrogen is ortho or meta relative to the point of attachment of R² to the imidazotriazinyl of Formula Ia.

20. The compound of claim 19, wherein R² is substituted by one or two groups independently selected from —Cl, —CN, —CH₃, and —CD₃.

21. The compound of claim 20 wherein R² is selected from:

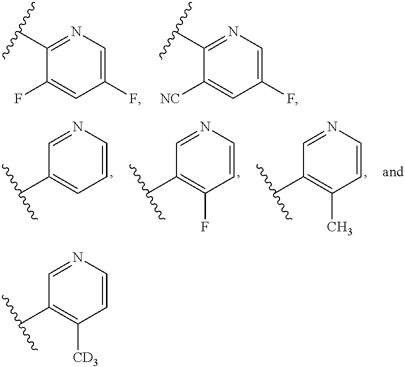

22. The compound of claim 7, wherein the compound is selected from any one of the following compounds: 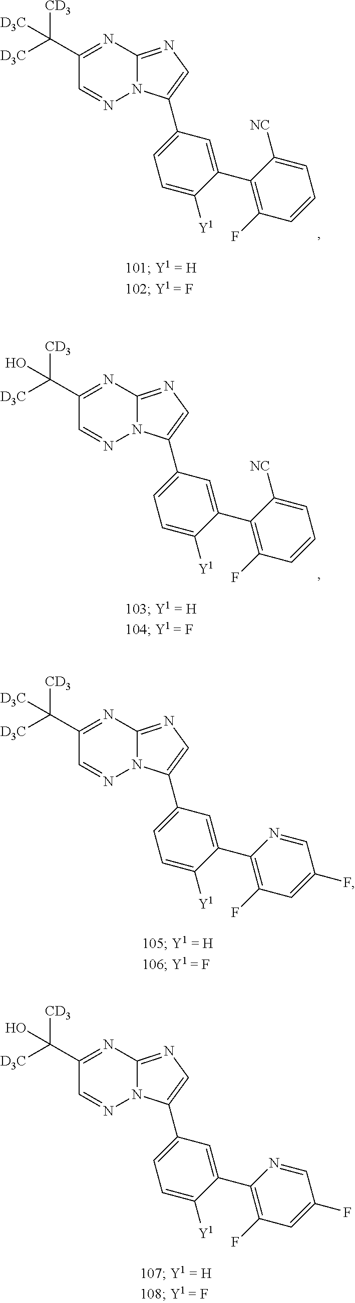 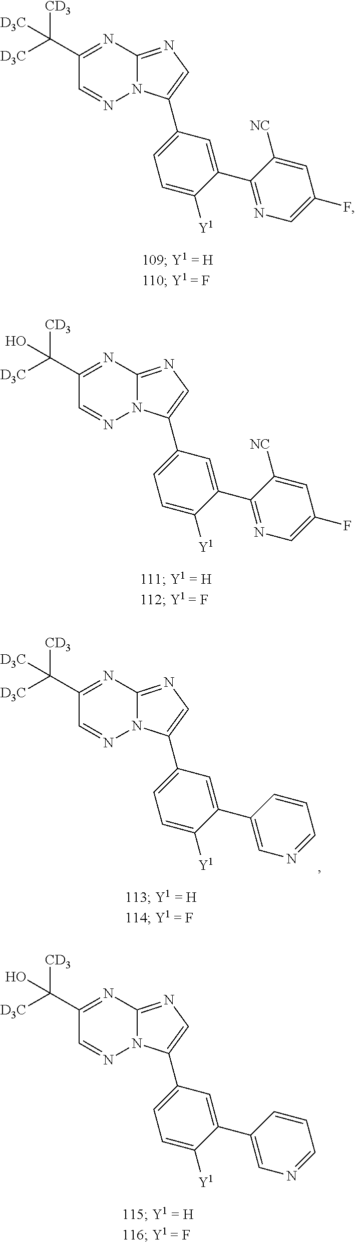 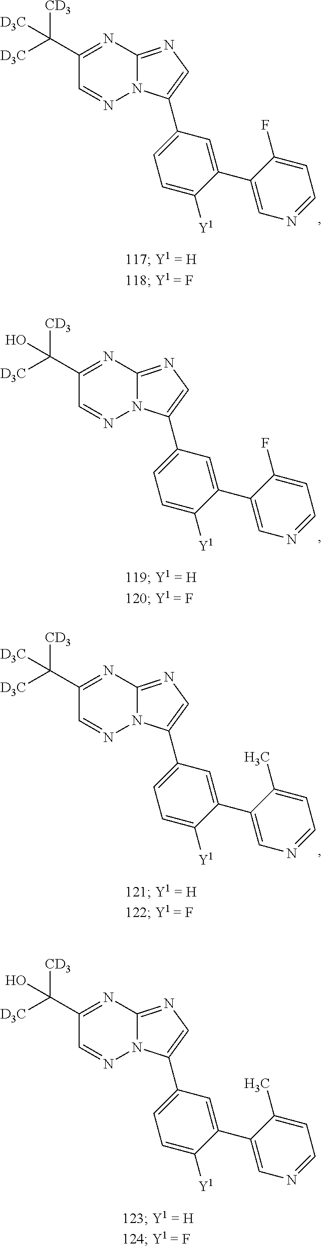 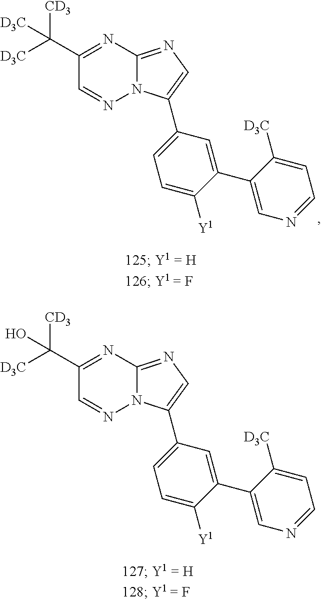
or a pharmaceutically acceptable salt of any of the foregoing.

23. A compound of formula II: 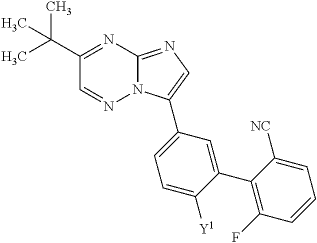
or a pharmaceutically acceptable salt thereof wherein Y¹ is hydrogen or —F.

24. The compound of claim 1, wherein any atom not designated as deuterium in any of the embodiments set forth above is present at its natural isotopic abundance.

25. A pyrogen-free pharmaceutical composition comprising a compound of claim 1 or a pharmaceutically acceptable salt of said compound; and a pharmaceutically acceptable carrier.

26. (canceled)

27. A method of treating a disease or condition selected from anxiety, convulsions, skeletal muscle spasm, spasticity, athetosis, epilepsy, stiff-person syndrome, other disorders of the central nervous system, and pain, in a subject comprising the step of administering to the subject an effective amount of a composition of claim 25.

28. The method of claim 27, wherein the disease or condition is anxiety or convulsions.

29. (canceled)

30. The method of claim 27, wherein the pain is selected from the group consisting of acute, chronic, neuropathic, or inflammatory pain, arthritis, migraine, cluster headaches, trigeminal neuralgia, herpetic neuralgia, general neuralgias, visceral pain, osteoarthritis pain, postherpetic neuralgia, diabetic neuropathy, radicular pain, sciatica, back pain, head pain, neck pain, severe or intractable pain, nociceptive pain, breakthrough pain, postsurgical pain, and cancer pain.

31. The method of claim 27, wherein the pain is selected from the group consisting of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, including abdominal, pancreatic, and IBS pain; chronic and acute headache pain; migraine; tension headache, including cluster headaches; chronic and acute neuropathic pain, including post-herpetic neuralgia; diabetic neuropathy; HIV-associated neuropathy; trigeminal neuralgia; Charcot-Marie Tooth neuropathy; hereditary sensory neuropathies; peripheral nerve injury; painful neuromas; ectopic proximal and distal discharges; radiculopathy; chemotherapy induced neuropathic pain; radiotherapy-induced neuropathic pain; post-mastectomy pain; central pain; spinal cord injury pain; post-stroke pain; thalamic pain; complex regional pain syndrome; phantom pain; intractable pain; acute pain, acute post-operative pain; acute musculoskeletal pain; joint pain; mechanical low back pain; neck pain; tendonitis; injury/exercise pain; acute visceral pain, including abdominal pain, pyelonephritis, appendicitis, cholecystitis, intestinal obstruction, and hernias; chest pain, including cardiac pain; pelvic pain; renal colic pain; acute obstetric pain, including labor pain; cesarean section pain; acute inflammatory, burn and trauma pain; acute intermittent pain, including endometriosis; acute herpes zoster pain; sickle cell anemia; acute pancreatitis; breakthrough pain; orofacial pain including sinusitis pain and dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; painful bladder syndrome; interstitial cystitis (IC); prostatitis; complex regional pain syndrome (CRPS), type I and type II; and angina-induced pain.

32. The method of claim 31, wherein the pain is selected from the group consisting of fibromyalgia, acute herpes zoster pain, HIV-associated neuropathy, neuropathic low back pain, chemotherapy induced neuropathic pain, radiotherapy-induced neuropathic pain, peripheral nerve injury, spinal cord injury pain, and multiple sclerosis (MS) pain.
